(19) United States
(12) Patent Application Publication
(10) Pub. No.: US 2013/0007910 A1
(43) Pub. Date: Jan. 3, 2013

(54) STRUCTURE OF THE BRASSINOSTEROID RECEPTOR BRI1, AND MODULATION OF BRI1 SIGNALING

(75) Inventors: Michael Hothorn, Lauenau (DE); Joanne Chory, Del Mar, CA (US)

(73) Assignee: Salk Institute for Biological Studies (027731), La Jolla, CA (US)

(21) Appl. No.: 13/473,426

(22) Filed: May 16, 2012

Related U.S. Application Data
(60) Provisional application No. 61/487,120, filed on May 17, 2011.

(51) Int. Cl. C12N 9/12; G06F 19/16; G01N 33/566

(52) U.S. Cl. 800/278; 435/194; 435/7.8; 703/11

(57) ABSTRACT

Provided herein is the crystal structure for the brassinosteroid receptor BRI1, as well as strategies for modulating its activity.

### CROSS-REFERENCES TO RELATED APPLICATIONS

The present application claims priority to U.S. Provisional Patent Application No. 61/487,120 filed May 17, 2011, the disclosure of which is incorporated herein by reference in its entirety.

### STATEMENT AS TO RIGHTS TO INVENTIONS MADE UNDER FEDERALLY SPONSORED RESEARCH AND DEVELOPMENT

The invention was made with Government support under Grant No. IOS-0649389 awarded by the National Science Foundation, and Grant Nos. AI042266 and P30 NS057096 from the National Institutes of Health. The Government has certain rights in the invention.

### BACKGROUND OF THE INVENTION

Polyhydroxylated steroids are regulators of body shape and size in higher organisms. In metazoans intracellular receptors recognize these molecules. Plants however perceive steroids at membranes, using the membrane-integral receptor kinase BRI1.

Signal perception at the cell surface and transduction of this signal to the cell interior is essential to all life forms. Plants have met this challenge in part by evolving membrane-integral receptor kinases (RKs). Many RKs are comprised of an extracellular Leucine-Rich Repeat (LRR1) (Kobe & Deisenhofer, Nature 366, 751-756 (1993)) module and a cytoplasmic kinase domain, connected by a single membrane-spanning helix (Shiu & Bleecker, Proc. Natl. Acad. Sci. U.S.A. 98, 10763-10768 (2001)). Receptors with this architecture (LRR-RKs) initiate signaling pathways that, for example, regulate plant growth (Li & Chory, Cell 90, 929-938 (1997)), development (Clark et al., Cell 89, 575-585 (1997); Nadeau & Sack, Science 296, 1697-1700 (2002)) and interactions with the environment (Gómez-Gómez & Boller, Mol. Cell. 5, 1003-1011 (2000); Zipfel et al., Cell 125, 749-760 (2006); Nishimura et al., Nature 420, 426-429 (2002)). The corresponding ligands range from small molecules (Wang et al., Nature 410, 380-383 (2001)) and peptides (Ogawa et al., Science 319, 294 (2008)); Sugano et al., Nature 463, 241-244 (2010)) to entire proteins.

The LRR-RK BRASSINOSTEROID INSENSITIVE 1 (BRI1) (Li & Chory; Belkhadir & Chory Science 314, 1410-1411 (2006)) controls a steroid signaling pathway essential for plant growth (Vert et al., Annu. Rev. Cell Dev. Biol 21, 177-201 (2005)). While animal steroid receptors are found predominantly in the nucleus (Mangelsdorf et al., Cell 83, 835-839 (1995)), BRI1 is localized at the plasma-membrane and in endosomes (Geldner et al., Genes Dev. 21, 1598-1602 (2007)).

The following model has been proposed for BRI1 activation. In the absence of brassinosteroid, BRI1's kinase domain is kept in a basal state by its auto-inhibitory C-terminal tail, as well as by interaction with the inhibitor protein BKI1. Hormone binding to the extracellular domain of BRI1 (Wang et al., Nature 410, 380-383 (2001); He et al., Science 288, 2360-2363 (2000)) in a region that includes a ˜70 amino acid ‘island’ domain between LRRs 21 and 22 (Kinoshita et al., Nature 433, 167-171 (2005)), causes a change in the receptor (a conformational change in a preformed homodimer (Wang et al., Dev. Cell 8, 855-865 (2005)) or receptor dimerization), leading to autophosphorylation of the BRI1 kinase domain (Wang et al., Plant Cell 17, 1685-1703 (2005)), release of its C-terminal tail and trans-phosphorylation of the inhibitor BKI1 (Wang & Chory, Science 313, 1118-1122 (2006); Jaillais et al., Genes Dev. 25, 232-237 (2011)). BKI1 then dissociates from the membrane, allowing BRI1 to interact with a family of smaller LRR-RKs (Chinchilla, Trends Plant Sci. 14, 535-541 (2009)), including the BRI1 ASSOCIATED KINASE 1 (BAK1). The kinase domains of BRI1 and BAK1 trans-phosphorylate each other on multiple sites (Wang et al., Dev. Cell 15, 220-235 (2008)), and the fully activated receptor triggers downstream signalling events (Kim & Wang, Annu. Rev. Plant Biol. 61, 681-704 (2010)), resulting in major changes in nuclear gene expression.

BRI1 is reminiscent of animal Toll-like innate immunity receptors (TLRs). Indeed several members of the plant LRR-RK family are innate immunity receptors. It was thus expected that the BRI1 ectodomain would form a TLR-like horseshoe structure (Choe et al., Science 309, 581-585 (2005)), and that BRI1 would bind its ligand along a dimer interface, like the TLRs (Liu et al., Science 320, 379-381 (2008); Park et al., Nature 458, 1191-1195 (2009)).

Reported herein is the structure of the ligand binding domain of BRI1 in its free form, and bound to the plant steroid brassinolide. The results show that, unlike TLRs, BRI1 folds into a superhelical assembly, whose interior provides the hormone-binding site. Comparison of the free and hormone-bound structures, combined with genetic data, suggests a novel activation mechanism for BRI1 that is distinct from TLRs.

### BRIEF SUMMARY OF THE INVENTION

Provided herein is the structure of BRI1, in both unbound and brassinolide-bound forms. The structure can be used to design novel synthetic brassinosteroid hormones or hormone mimetics with unique properties. In addition, the interactions within the brassinolide-BRI1 complex allow for rational design of modified, e.g., labeled or stabilized, forms of brassinosteroid without affecting the interactions within the complex. The structure also reveals sites on BRI1 that can be used to design BRI1 antagonists. For example, now that the ligand binding and interaction sites are defined, antagonist compounds that can, e.g., block or interfere with ligand or co-receptor binding can be designed. In some embodiments, such antagonists can be used as herbicides or to control the timing of plant growth and development.

Accordingly, in some embodiments, provided herein is an isolated protein comprising a 3-dimensional crystal structure of a BRassinosteroid Insensitive 1 (BRI1) ectodomain as structurally defined by the atomic coordinate data shown in Tables 1 and 2. In some embodiments, the isolated protein comprises a 3-dimensional structure of the BRI1 ectodomain, with a space group C2 and unit cell dimensions a=175.09±0.1 angstrom, b=67.25±0.1 angstrom, c=119.05±0.1, with beta=121.55±0.1. In some embodiments, the isolated protein comprises a 3-dimensional structure of the BRI1 ectodomain as structurally defined by the diagrams shown in FIG. 1 and FIG. 9. In some embodiments, the protein comprises a homolog of the Arabidopsis BRI1 ectodomain sequence shown in SEQ ID NO:1, i.e., an ortholog from a different species or a paralog from Arabidopsis. In some embodiments, the protein comprises a sequence having at least 90% identity to residues 29-788 of SEQ ID NO:1. In some embodiments, the protein binds brassinolide. In some embodiments, the protein interacts with BAK1 (Brassinosteroid Associated Kinase 1).

In some embodiments, provided are methods for identifying (screening for) a candidate modulator of BRI1, comprising
(a) comparing the structure of a test compound with the structure of BRI1, said BRI1 comprising a 3 dimensional structure selected from the group consisting of:
   (i) an ectodomain structurally defined by the atomic coordinate data shown in Tables 1 and 2;
   (ii) a space group C2 and unit cell dimensions a=175.09±0.1 angstrom, b=67.25±0.1 angstrom, c=119.05±0.1, with beta=121.55±0.1; and
   (iii) an ectodomain structurally defined by the diagrams shown in FIG. 1 and FIG. 9;
(b) determining whether the test compound is likely to interact with BRI1; and
(c) identifying a candidate BRI1 modulator when the test compound in step (b) is determined to be likely to interact with BRI1.

In some embodiments, the method further comprises validating the candidate BRI1 modulator by contacting the candidate BRI1 modulator with BRI1 and detecting interaction of the candidate BRI1 modulator with BRI1. In some embodiments, the method further comprises detecting an effect of the candidate BRI1 modulator when contacted with a BRI1 expressing plant, wherein the effect is selected from the group consisting of increasing or decreasing plant biomass and increasing or decreasing the size of vegetative structures in the plant, as compared to a standard control. In some embodiments, the candidate BRI1 modulator interacts with the ligand binding region of BRI1 (e.g. within LRR 21-25). In some embodiments, the candidate BRI1 modulator interacts with the co-receptor interaction region of BRI1 (e.g., within LRR 21-25 and the island domain, see FIG. 13).

In some embodiments, provided are methods for identifying (screening for) a candidate modulator of BRI1, comprising
(a) contacting a test compound with BRI1, said BRI1 comprising a 3 dimensional structure selected from the group consisting of:
   (i) an ectodomain structurally defined by the atomic coordinate data shown in Tables 1 and 2;
   (ii) a space group C2 and unit cell dimensions a=175.09±0.1 angstrom, b=67.25±0.1 angstrom, c=119.05±0.1, with beta=121.55±0.1; and
   (iii) an ectodomain structurally defined by the diagrams shown in FIG. 1 and FIG. 9; and
(b) detecting interaction of the test compound with BRI1, thereby identifying a candidate modulator of BRI1.

In some embodiments, the method further comprises rational design of the test compound, e.g., based on the BRI1 structure described herein. For example, prior to step (a), the structure of a test compound can be compared to the structure of BRI1 to determine the likelihood of interaction between the test compound BRI1. In some embodiments, the method further comprises detecting an effect of the candidate BRI1 modulator when contacted with a BRI1 expressing plant, wherein the effect is selected from the group consisting of: increasing or decreasing plant biomass and increasing or decreasing the size of vegetative structures in the plant, as compared to a standard control. In some embodiments, the candidate BRI1 modulator interacts with the ligand binding region of BRI1 (e.g. within LRR 21-25). In some embodiments, the candidate BRI1 modulator interacts with the co-receptor interaction region of BRI1 (e.g., within LRR 21-25 and the island domain, see FIG. 13).

Also provided are BRI1 modulators. In some embodiments, the BRI1 modulator is identified according to a method as described above. In some embodiments, the BRI1 modulator is brassinosteroid mimetic identified as likely to bind the brassinosteroid binding site of BRI1 as characterized herein (e.g., in FIG. 9, Tables 1 and 2). In some embodiments, the BRI1 modulator interacts with BRI1 in the same way as brassinolide (i.e., at the same residues and/or with the same affinity), which is included as a BRI1 modulator. In some embodiments, the BRI1 modulator interacts with BRI1 in a manner that is distinct from brassinolide (i.e., at different residues and/or with a higher or lower affinity). In some embodiments, the BRI1 modulator is modified, e.g., with a label, or to improve stability, using the BRI1 structure described herein to ensure that the modification does not interfere with the BRI1 interaction.

In some embodiments, the BRI1 modulator is a BRI1 inhibitor. In some embodiments, the BRI1 inhibitor interferes with, e.g., ligand binding to BRI1 or co-receptor interaction with BRI1. Such inhibitors can be designed using the BRI1 structural data disclosed herein, e.g., to target BRI1 residues critical for BRI1 ligand binding or co-receptor interaction (see, e.g., FIGS. 9, 18 and Table 2). In some embodiments, the BRI1 inhibitor, when contacted with a plant expressing BRI1, reduces plant biomass and/or reduces the size of vegetative structures (e.g., stems, leaves, etc.) as compared to a standard control (e.g., a BRI1 expressing plant in the absence of the inhibitor).

In some embodiments, the BRI1 modulator is a BRI1 agonist. Such agonists can be designed using the BRI1 structural data disclosed herein, e.g., to target BRI1 residues involved in binding to the natural BRI1 ligand or co-receptor (see, e.g., FIGS. 9, 18 and Table 2). In some embodiments, the BRI1 agonist mimics or improves the binding of a brassinosteroid (such as brassinolide) to BRI1. In some embodiments, the BRI agonist stabilizes the co-receptor interaction domain, stabilizes the interaction between BRI1 and a co-receptor (e.g., BAK or BAK-like proteins). In some embodiments, the BRI1 agonist, when contacted with a plant expressing BRI1, increases plant biomass and/or increases the size of vegetative structures, as compared to a standard control (e.g., a BRI expressing plant absent the agonist).

### BRIEF DESCRIPTION OF THE DRAWINGS

FIG. 1. The BRI1 ectodomain forms a superhelical assembly. a, Ribbon diagram of the BRI1 LRR domain (front, back and top). The canonical LRR β-sheet is shown in orange, and the additional plant-specific β-sheets in blue. Helices are shown in green, and the island domain is depicted in red. b, Structural comparison of the BRI1 (shown as yellow C_{α} trace) and TLR3 (in blue, pdb-id: lziw) (Choe et al., Science 309, 581-585 (2005)) ectodomains. The structures superimpose with an r.m.s.d. of 4.2 Å between 341 corresponding C_{α} atoms. Side and top views are shown. The island domain has been omitted for clarity.

FIG. 2. Single Isomorphous Replacement phasing of the BRI1 ectodomain using a sodium iodide shortsoak. a, Stereo view of a C_{α} trace of the BRI1 structure (in blue) shown together with the 26 heavy atom sites (in yellow) identified with the programs SHELXD (Sheldrick, Crystallogr. 64, 112-122 (2008)) and SHARP (Bricogne et al., Acta Crystallogr. D Biol. Crystallogr. 59, 2023-2030 (2003)) that were used for SIRAS phasing (min/max/mean refined occupancies are 0.1/0.57/0.33). b, Stereo view of the initial 2F_{O}-F_{C} electron density map (contoured at 1.5 σ) obtained after density modification and phase extension to 2.5 Å using the program phenix.resolve (Terwilliger et al., Acta Crystallogr. D Biol. Crystallogr. 64, 61-69 (2008)).

FIG. 3. The BRI1 ectodomain is structurally related to bacterial LRR proteins and to the plant defense protein PGIP. Structural comparison of the BRI1 ectodomain (shown as C_{α} trace in yellow; the island domain has been omitted for clarity) and the LRR domains of a, the extracellular bacterial effector protein YopM (Evdokimov et al., J. Mol. Biol. 312, 807-821 (2001)) (shown in blue, r.m.s.d. is 3.0 Å between 313 corresponding C_{α} atoms, DALI (Holm et al., Bioinformatics 24, 2780-2781 (2008)) Z-score is 20.0). b, the bacterial adhesion protein internalin A (Schubert et al., Cell 111, 825-836 (2002)) (r.m.s.d. is 2.2 Å between 291 corresponding C_{α} atoms, DALI Z-score is 21.6), and c, the polygalacturonase-inhibiting protein (PGIP) from Phaseolus vulgaris (Di Matteo et al., Proc. Natl. Acad. Sci. U.S.A. 100, 10124-10128 (2003)) (r.m.s.d. is 2.3 Å between 252 corresponding C_{α} atoms, DALI Z-score is 19.8).

FIG. 4. The plant LRR proteins PGIP and BRI1 contain additional β-sheets that cause supertwisting of their LRR domains. Stereo view of a structural superposition of the BRI1 ectodomain and the polygalacturonase-inhibiting protein (PGIP) from Phaseolus vulgaris (Evdokimov et al., J. Mol. Biol. 312, 807-821 (2001)) (r.m.s.d. is 2.3 Å between 252 corresponding C_{α} atoms). Both proteins are shown in ribbon representation, PGIP in yellow, BRI1 in light-blue. Note that the non-canonical β-sheet in PGIP (shown in dark-orange) that causes twisting of the PGIP LRR domain, is also present in BRI1 over the entire length of the molecule (shown in dark blue).

FIG. 5. Plant-specific sequence fingerprints result in a superhelical BRI1 ectodomain. a, Ribbon diagram of the convex side of BRI1 LRRs 9-25 (in yellow). The non-canonical β-strands and the Ile-Pro spine are shown in dark and light blue, respectively. b, Top view of the BRI1 ectodomain (in blue) with disulfide bridges shown in yellow. The N- and C-terminal caps are highlighted in pink. c, Sequence alignment of LRRs in BRI1, other plant receptor kinases (Clark et al., Cell 89, 575-585 (1997); Nadeau & Sack, Science 296, 1697-1700 (2002); Gómez-Gómez & Boller, Mol. Cell. 5, 1003-1011 (2000); Zipfel et al., Cell 125, 749-760 (2006); Nam & Li, Cell 110, 203-212 (2002)) and PGIP. The canonical LRR consensus sequences are highlighted in blue, and plant-specific motifs are in green.

FIG. 6. Key sequence fingerprints of the BRI1 ectodomain are conserved in BRI1 proteins from different plant species. Structure-based sequence alignment of representative BRI1 orthologs: Arabidopsis thaliana BRI1 UniProt (at the website found at uniprot.org): 022476, residues 29-771), (Solarium lycopersicum (UniProt: Q8GUQ5, residues 37-780), Glycine max (UniProt: C6FF79, residues 21-765), Nicotiana tabacum (UniProt: A6N8J1, residues 46-787), Oryza sativa subsp. japonica (UniProt: Q942F3, residues 21-696), Arabidopis thaliana BRI1-like 3 (UniProt: Q9LJF3, residues 27-765). The alignment includes secondary structure assignments with DSSP (Kabsch & Sander Biopolymers 22, 2577-2637 (1983)), coloured according to FIG. 1a. Cysteine residues in the LRRs and in the N- and C-terminal capping domains that form disulfide bonds are highlighted by yellow shading, N-glycosylation sites observed in the BRI1 structure are depicted with red letters. The positions of known missense alleles in the BRI1 ectodomain are indicated with blue lettering.

FIG. 7. The BRI1 structure identifies plant-specific capping motifs. a, Ribbon diagram of the N-terminal capping structure, and b, of the C-terminal capping motif. The amphipatic α-helix and the small 3₁₀ helices are shown in blue, and β-strands are depicted in orange. Conserved interfacing residues are highlighted in full atom representation. Mutation of Cys69 into Tyr (the genetic allele bri1-5) may destabilise the N-terminal cap in BRI1 and thus causes the receptor to be retained in the endoplasmic reticulum (Noguchi et al., Plant Physiol. 121, 743-752 (1999); Belkhadir et al., Genetics 185, 1283-1296 (2010)). Superimposed in green are the corresponding caps from the plant defense protein PGIP that closely align with the BRI1 structure. Structure based sequence alignments that depict the corresponding capping motifs in the LRR-RKs BAK1 (Li et al., Cell 110, 213-222 (2002), CLV1 (Clark et al., Cell 89, 575-585 (1997)), EFR, FLS2, and TMM indicate that similar capping structures are present in other plant receptor kinases.

FIG. 8. The island domain makes intensive contacts with the C-terminal LRR motifs in BRI1. Stereo view of LRRs 13-25 (ribbon diagram) in blue and the island domain (residues 584-654) in orange. Interface residues originating from the LRR core and the island domain are shown in light blue and yellow, respectively. Polar interactions (distance cut-off 3.5 Å) are depicted as dotted lines (in red). The BRI1 loss-of-function mutations Gly611Glu (bri1-113) (Li & Chory, Cell 90, 929-938 (1997)) and Ser662Phe (bri1-9) are highlighted by magenta balls.

FIG. 9. The steroid hormone binding site maps to C-terminal inner surface of the superhelix. a, Brassinolide (in yellow sticks) binds to a surface provided by the LRR domain (in blue) and by parts of the island domain (green ribbon). b, Location of the steroid in centre of the BRI1 superhelix. c, Close-up view of the brassinolide in two orientations, including an omit 2Fₒ-F_{c} electron density map contoured at 1.5 σ. d, Protein-hormone interactions in the BRI1 steroid binding site. Ribbon diagram of LRRs 21-25 (in grey) are shown together with parts of the island domain (in green). Contacting residues are shown in full side-chain representation, polar interactions as dotted lines, and water molecules as red balls. Bri1-6 (Gly644Asp) is depicted in magenta.

FIG. 10 The island domain and the two connecting loops become fully ordered upon steroid hormone binding. Structural superposition of the free and brassinolide-bound ectodomain reveal no major conformational changes (r.m.s.d. <0.3 Å comparing 740 corresponding C_{α} atoms), but the entire island domain appears to be significantly better ordered in the steroid-complex when compared to the free structure. C_{α} trace views of the free and brassinolide bound structures colored according to their crystallographic B-values (low (15 Å²) to high (150 Å²) corresponding to blue and red, respectively). The island domains are highlighted, and the steroid ligand is shown in sticks representation (in yellow). The average B-values for the LRR and island domains are 59.5 and 93.1 Å² (free from) and 64.4 and 65.5 Å² (brassinolide complex), respectively. Both the free and the steroid bound form crystallized under similar conditions, in the same space-group and with similar cell constants; and both diffracted to ˜2.5 Å resolution.

FIG. 11. Chemical structures of steroid ligands. Chemical structures of the plant steroids a, brassinolide and b, castasterone and c, of the arthropod ecdysone. The ring nomenclature for brassinolide has been included in a.

FIG. 12. N-linked glycans mask large surface areas of the BRI1 superhelix. Oligomannose core structures (containing two N-acetylglucosamines and three mannose units) as found in insect cells and plants were modeled onto the nine glycosylation sites in the structure to visualise the BRI1 surface that is potentially masked by carbohydrate. The LRR domain in surface representation is shown in blue, and the glycan structures are highlighted in yellow. A ribbon diagram of the island domain (in green) and the steroid ligand (in yellow) is included. The views are a, front b, back and c, perspective along the superhelix from the C-terminus.

FIG. 13. An accessible membrane-proximal region of BRI1 can provide a protein-protein interaction platform. a, Overview of the C-terminal surface area (in blue) that is not masked by carbohydrate. Brassinolide is shown in yellow, the island domain in orange, and genetic alleles connected in magenta. b, Analytical gel-filtration 280 nm absorbance trace. The free ectodomain eluates as a monomer (black line), as does a putative complex with brassinolide (red line). Void (V₀) and total volume (V₁) are shown together with elution volumes for molecular weight standards (A, aldolase, MW 158,000 Da; B, conalbumin, MW 75,000 Da). The estimated molecular weight for the monomer peak is ˜125 kDa. The approximate molecular weight of the purified BRI1 is 110 kDa.

FIG. 14. Genetic BRI1 missense alleles may affect the positioning of an island domain loop. Close up view of the three genetic alleles that are located in a loop segment (residues 643 to 658) connecting the island domain with LRR 22. Gly643 that in the genetic allele sudl is mutated to Glu, may engage in a hydrogen bond with Ser623 in the island domain. This would restrict movement of the loop segment and thus stabilize interaction with a co-receptor protein even in absence of steroid. Mutation of Gly644 into Asp causes the loss-of-function phenotype bri1-6, and mutation of the conserved Thr649 to Lys inactivates barley BRI1. These mutations appear to induce steric clashes with residues in the island domain and in the underlying LRR domain (indicated by black arrows) and thus distorts the overall position of the loop.

FIG. 15. The superhelical BRI1 ectodomain is unlikely to dimerize. Model of a BRI1 ectodomain homodimer in surface representations (top panel) and as a C_{α} trace (lower panel), with a front view on the left and a view from the top on the right. The model brings the C-termini of two neighboring ectodomains (shown in blue, and yellow, respectively) in close proximity and employs the proposed protein interaction platform and the brassinosteroid ligand (in red) as the dimerization interface. Because of the superhelical shape of the ectodomain, this model causes severe clashes in the N-terminal LRRs of BRI1 (indicated by arrows).

FIG. 16. Crystal lattice packing analysis indicates the BRI1 ectodomain is monomeric. a, C_{α} trace of the major lattice contact observed in the monoclinic BRI1 crystals. The interface brings two neighboring molecules in a head-to-head configuration with their C-termini far apart at opposite ends. b, View of the second minor crystal contact that involves interaction of two BRI1 molecules with their outer helix surfaces along the 2-fold symmetry axis. c, Crystal packing of BRI1 molecules along a. The BRI1 superhelix propagates itself using the head-to-head contacts described in a.

FIG. 17. Homology model of the BAK1 ectodomain a, Ribbon diagram of a homology model of the BAK1 ectodomain based on the crystal structures of BRI1 (this study) and PGIP with the program MODELLER. This model suggests that the N-terminal residues 27-69 do not form a leucine zipper motif as previously suggested, but an N-terminal capping motif that is highly similar to that in BRI1 and PGIP (see FIG. 7). The present BAK1 model contains 5 repeats that contain the canonical β-strand (in orange), followed by the non-canonical β-strand found in the plant-specific LRR subfamily (Kajava, J. Mol. Biol. 277, 519-527 (1998)) (in blue), and a 3₁₀ helix (in green). The BAK1 elg allele (Halliday et al., Plant J. 9, 305-312 (1996)), which renders mutant plants hypersensitive to brassinosteroid treatment (Whippo & Hangarter, Plant Physiol. 139, 448-457 (2005)), maps to the inner surface of the BAK1 ectodomain. The C-terminal capping motif is a proline-rich motif that in sequence deviates from the C-terminal capping motifs found in BRI1 and PGIP (see FIG. 7), and therefore could not be modelled with confidence (in grey). b, The family-defining sequence fingerprints of the plant-specific LRR subfamily (Lt/sGxIP) are present in all 5 LRR repeats in BAK1 (in green). The position of the elg mutation is indicated in red.

FIG. 18. Model for BRI1 receptor activation by heteromerization with the smaller co-receptor kinase BAK1. Side-by-side view of the BRI1 ectodomain structure (surface representation, in dark-blue) and the BAK1 homology model (in light-blue) indicates that the BAK1 ectodomain is compatible in size and shape with the protein interaction surface in BRI1. In this model, steroid binding to BRI1 generates a docking platform for the ectodomain of BAK1. This docking platform in BRI1 is composed of the steroid ligand itself, of parts of the island domain (in orange) (especially the connecting loops that become ordered upon steroid binding, in magenta), and of surface patches of the LRR domain (i.e., Thr750, whose mutation to isoleucene causes the loss-of-function phenotype 102 (Friedrichsen, Plant Physiol. 123, 1247-1256 (2000)), in magenta). Steroid-dependent heteromerization of the BRI1 and BAK1 ectodomains brings their cytoplasmic juxtamembrane regions and kinase domains in close proximity, where they transphosphorylate each other, leading to receptor activation and phosphorylation of downstream signaling partners. This mechanism is plausible even if BRI1 forms constitutive homooligomers at plant membranes, as long as the interaction surfaces for BAK1 are accessible in these oligomers. The data indicate that the gain-of-function alleles sud1 (Diévart, Funct. Plant Biol. 33, 723-730 (2006)) in BRI1 and elg (Halliday et al., Plant J. 9, 305-312 (1996); Whippo & Hangarter, Plant Physiol. 139, 448-457 (2005)) in BAK1 stabilize formation of the heteromeric complex, while loss-of-function alleles 6 (Noguchi et al., Plant Physiol. 121, 743-752 (1999)), 102 (Friedrichsen, Plant Physiol. 123, 1247-1256 (2000)) and the mutation in barley BRI1 (Gruszka, J. Appl. Genet. (2011)) (Thr649 in AtBRI1) (all in magenta) destabilize interaction of the BAK1 and BRI1 ectodomains.

### DETAILED DESCRIPTION OF THE INVENTION

### I. Introduction

Provided herein is the BRI1 ectodomain structure at 2.5 angstrom resolution. The structure reveals a superhelix of 25 twisted leucine-rich repeats (LRRs), an architecture that is strikingly different from the assembly of LRRs in animal Toll-like receptors. A 70 amino-acid island domain between LRRs 21 and 22 folds back into the interior of the superhelix to create a surface pocket for binding the plant hormone brassinolide. Known loss- and gain-of-function mutations closely map to what is herein revealed to be the hormone-binding site. The structure described herein indicates that steroid binding to BRI1 generates a docking platform for a co-receptor that is required for receptor activation. The findings have mechanistic implications for hormone, developmental, and innate immunity signaling pathways in plants that use similar receptors.

The structure of the BRI1 ectodomain offers several new insights, and its twisted shape will likely characterize the architecture of many plant LRR-RKs. The presence of a folded domain as an LRR insertion is likely an adaptation to recognize a small molecule ligand, a challenge that smaller LRR proteins have met by generating loop insertions into their capping motifs (Han et al., Science 321, 1834-1837 (2008)). The unusual superhelical structure of BRI1 and its fascinating mode of ligand recognition provides insights into how steroids can be sensed at membranes and rationalizes a large set of genetic and biochemical findings.

The structure of the BRI1-brassinolide complex is informative about the molecular interactions between the ligand and receptor. This for the first time explains why certain chemical modifications of brassinolide are tolerated, while others are inactivated, and why animal steroid hormones with similar structure are not effective in plants (see, e.g., Back and Pharis (2003) J. Plant Growth Regul. 22:350.

In addition, comparison of the brassinolide-bound and unbound structures are informative of the mechanism of activation. Brassinolide binding creates a docking platform in BRI1, which is recognized by the extracellular LRR domain of BRI1 Associated Kinase 1 (BAK1). BAK1 thus can act as a co-receptor for activation of the BRI1 signaling pathway.

### II. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N.Y. 1989); Raven et al. PLANT BIOLOGY (7^{th} ed. 2004). Any methods, devices and materials similar or equivalent to those described herein can be used in the practice of this invention. The following definitions are provided to facilitate understanding of certain terms used frequently herein and are not meant to limit the scope of the present disclosure.

The term “BRI1” refers to “Brassinolide Insensitive 1” proteins, homologs (e.g., orthologs from different species or paralogs), BRI1 fragments having BRI1 activity, and BRI1 variants having substantial identity to a naturally occurring BRI1 with BRI1 activity. BRI1 activities include, e.g., brassinosteroid binding, phosphorylation of BAK1 and initiation of the BR signaling pathway, increasing plant mass, and increasing the size of vegetative structures.

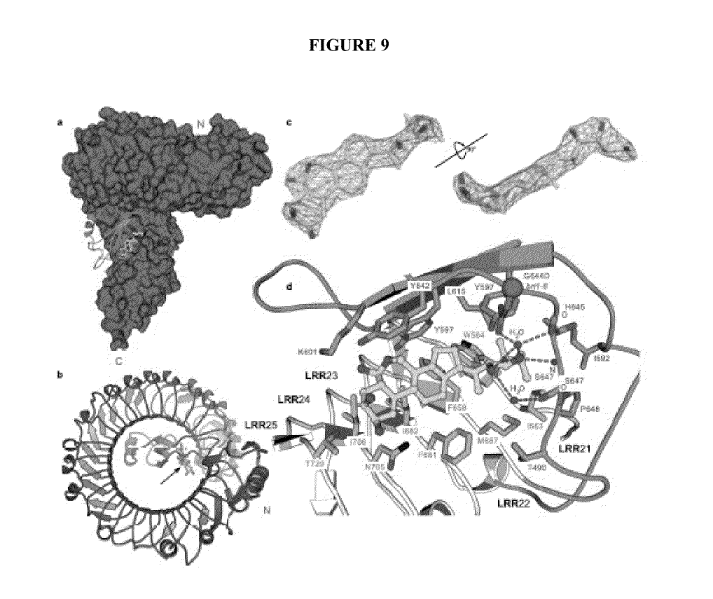

A “brassinosteroid analog” or “brassinosteroid mimetic” is a compound that has a similar structural interaction with BRI1 as a brassinosteroid. Brassinosteroid mimetics include “brassinolide analogs” and “brassinolide mimetics.” In some cases, the brassinosteroid mimetic also has brassinosteroid activity and activates BRI1 signaling. Brassinosteroid mimetics can be steroidal or non-steroidal, and include B-ring analogs, side chain analogs (e.g., C-3, C-24, C-25, and C-26 side chain analogs), and methyl ether analogues. Brassinosteroid mimetics also include compounds that are designed considering the BRI1 structure provided herein, e.g., to have a similar interaction with BRI1 as brassinolide, as shown in FIG. 9.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes, biotin, digoxigenin, or haptens and proteins or other entities which can be made detectable, e.g., by incorporating a radiolabel into a peptide or antibody specifically reactive with a target peptide. Any method known in the art for conjugating a compound or protein to the label may be employed, e.g., using methods described in Hermanson, Bioconjugate Techniques 1996, Academic Press, Inc., San Diego.

A “labeled” or “tagged” molecule (e.g., compound, modulator, protein, or antibody) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following amino acids are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by the local homology algorithm of Smith and Waterman, Add. APL. Math. 2:482 (1981), by the homology alignment algorithm of Needle man and Wunsch, J. Mol. Biol. 48:443 (1970), by the search for similarity method of Pearson and Lipman, Proc. Natl. Acad. Sci. U.S.A. 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” This definition also refers to, or may be applied to, the compliment of a nucleotide test sequence. The definition also includes sequences that have deletions and/or additions, as well as those that have substitutions. As described below, the preferred algorithms can account for gaps and the like. Preferably, identity exists over a region comprising a ligand binding site or interaction domain, or a sequence that is at least about 25 amino acids or nucleotides in length, or over a region that is 50-100 amino acids or nucleotides in length.

The term “recombinant” when used with reference, e.g., to an organism, cell, nucleic acid, protein, or vector, indicates that the organism, cell, nucleic acid, protein or vector has been modified by the introduction of a heterologous nucleic acid or protein or the alteration of a native nucleic acid or protein, or that the cell is derived from a cell so modified. Thus, for example, recombinant cells and organisms express genes that are not found within the native (non-recombinant) form of the cell or express native genes that are otherwise abnormally expressed, under expressed or not expressed at all.

A polynucleotide or polypeptide is “heterologous to” a second polynucleotide or polypeptide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from any naturally occurring allelic variants.

An “expression cassette” refers to a nucleic acid construct, which when introduced into a host cell, results in transcription and/or translation of a RNA or polypeptide, respectively. Antisense or sense constructs that are not or cannot be translated are included by this definition.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seeds (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, bryophytes, and multicellular algae. It includes plants of a variety of ploidy levels, including aneuploid, polyploid, diploid, haploid and hemizygous.

The term “specifically bind” refers to a compound (e.g., BRI1-binding compound) that binds to a target with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity.

The term “compete”, as used regarding a BRI1 ligand, modulator, or interacting protein, means that a first compound competes for binding to BRI1 with a second compound, where binding of the first compound to its site on BRI1 is detectably decreased in the presence of the second compound compared to the binding of the first compound in the absence of the second compound. The alternative, where the binding of the second compound to its site on BRI1 is also detectably decreased in the presence of the first compound, can, but need not be the case. That is, a first compound can inhibit the binding of a second compound to its site without that second compound inhibiting the binding of the first compound to its respective site. However, where each compound detectably inhibits the binding of the other to BRI1, whether to the same, greater, or lesser extent, the compounds are said to “cross-compete” with each other for binding of their respective site(s).

The term “modulator” includes inhibitors and activators. Inhibitors are agents that, e.g., inhibit expression or bind to, partially or totally block stimulation or down regulate the activity of the described target protein, e.g., BRI1. Activators are agents that, e.g., induce or activate the expression of a described target protein or bind to, stimulate, or up regulate the activity of described target protein, e.g., BRI1. Modulators include naturally occurring and synthetic ligands, antagonists and agonists (e.g., small chemical molecules, steroids, antibodies, etc. that affect target activity). Assays for inhibitors and activators include, e.g., applying candidate modulator compounds to cells expressing the described target protein (e.g., BRI1 expressing cells) and then determining the functional effects on the described target protein activity. Samples or assays comprising described target protein that are treated with a potential activator, inhibitor, or modulator are compared to control samples without the inhibitor, activator, or modulator to examine the extent of effect. Control samples (untreated with modulators) can be assigned a relative activity value of 100%.

The terms “agonist,” “activator,” “inducer” and like terms refer to molecules that increase activity or expression as compared to a control. Agonists are agents that, e.g., bind to, stimulate, increase, activate, enhance activation, sensitize or upregulate the activity of the target. The expression or activity can be increased 10%, 20%, 30%, 40%, 50%, 60%, 70%, 80%, 90% 100% or more than that of a control (i.e., 110%, 120%, etc.). In certain instances, the activation is 1.5-fold, 2-fold, 3-fold, 4-fold, 5-fold, 10-fold, or more in comparison to a control.

The terms “inhibitor,” “repressor” or “antagonist” or “downregulator” interchangeably refer to a substance that results in a detectably lower expression or activity level as compared to a control. The inhibited expression or activity can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control.

A “control” sample or value refers to a sample that serves as a reference, usually a known reference, for comparison to a test sample. For example, a test sample can be taken from a test condition, e.g., in the presence of a test compound (e.g., candidate BRI1 modulator), and compared to samples from known conditions, e.g., in the absence of the test compound (negative control), or in the presence of a known compound (positive control, e.g. brassinolide or other known BRI1 modulator). A control can also represent an average value gathered from a number of tests or results. One of skill in the art will recognize that controls can be designed for assessment of any number of parameters. Controls can be designed for in vitro applications, e.g., for comparison to the binding activity and location of various candidate BRI1 modulators. Controls can also be designed for in situ or in vivo applications, e.g., for comparison to the effect of candidate BRI1 modulators on a BRI1-expressing plant or plant part. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As described below in detail, we have crystallized the BRI1 ectodomain and island domain in unbound and brassinolide-bound forms (see, e.g., Tables 1 and 2). Several parameters can be used to uniquely describe the symmetry and geometrical characteristics of a crystal. These include the space group (symmetry), the three unit cell axial lengths “a”, “b”, and “c”, and the three unit cell interaxial angles “α”, “β”, and “γ” (geometry). “Unit cell axial length” and “unit cell interaxial angle” are terms of art that refer to the three-dimensional geometrical characteristics of the unit cell, in essence its length, width, and height, and whether the building block is a perpendicular or oblique parallelepiped. The unit cell axial lengths and interaxial angles can vary by as much as ±10% without substantively altering the arrangement of the molecules within the unit cell. Thus, reference to each of the unit cell axial lengths and interaxial angles as being “about” a particular value is to be understood to mean that any combination of these unit cell axial lengths and interaxial angles can vary by as much as ±10% from the stated values.

### III. Methods of Protein Expression

BRI1, BRI1 domains, BRI1 interacting proteins (e.g., BAK1, or BAK1-like proteins or domains), and the like can be recombinantly expressed according to methods known in the art (see, e.g., Mus-Vetaux, Heterologous Expression of Membrane Proteins (2009); Glorioso et al. Expression of Heterologous Genes in Eukaryotic Systems, Methods in Enzymology Vol. 306 (1999)).

The sequence and domains for BRI1 and BAK1 are publically available at the NCBI website (ncbi.nlm.nih.org) for several plant species. For example, the Arabidopsis Uniprot accession number for BRI1 is O22476 (see also SEQ ID NO:1). One of skill will understand that homologs (e.g., orthologs from other species or paralogs within the same species such as BRI3) can be optimally aligned so that conserved residues can be located on the respective BRI1 proteins (see, e.g., Holton et al. (2007) Plant Cell 19:1709; Cano-Delgado et al. (2004) Development 131:5341).

Provided herein are recombinant expression cassettes comprising a promoter sequence operably linked to a nucleic acid sequence encoding a desired polypeptide sequence (e.g., BRI1, BRI1 variants and species homologs, a BRI1 domain, a BRI1-interacting protein, etc.). In some embodiments, the BRI1 domain is an ectodomain. In some embodiments, the BRI1-interacting protein is BAK1.

To use isolated sequences in the above techniques, recombinant DNA vectors suitable for transformation of cells can be prepared. Techniques for transforming a wide variety of higher plant species are well known and described in the technical and scientific literature, e.g., Weising et al., Ann. Rev. Genet. 22:421-477 (1988). Methods for expression in insect cells are described in more detail in the examples. Any cell type can be used for overexpression and protein production, as will be familiar to one of skill in the art, and kits for protein expression and purification are commercially available (e.g., from Invitrogen). A DNA sequence coding for the desired polypeptide, for example a cDNA sequence encoding a full-length protein, can be combined with transcriptional and translational initiation regulatory sequences which will direct the transcription of the sequence from the gene in the intended cell. In the context of the present invention, protein expression for the purpose of in situ or in vivo functional studies is typically carried out in plant cells, plant tissues, or whole plants (transgenic plants).

For example, a plant promoter can be employed which will direct expression of the gene in all tissues of a regenerated plant. Such promoters are referred to herein as “constitutive” promoters and are active under most environmental conditions and states of development or cell differentiation. Alternatively, the plant promoter can direct expression of the polynucleotide of the invention in a specific tissue (tissue-specific promoters, organ-specific promoters) or specific environmental condition (inducible promoters).

A polyadenylation region at the 3′-end of the coding region can be included. The polyadenylation region can be derived from the natural gene, from a variety of other plant genes, or from T-DNA.

The vector comprising the sequences (e.g., promoters or coding regions) from genes of the invention will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

Coding sequences, e.g., nucleic acid sequences that encode the BRI1 protein, can expressed recombinantly in plant cells. A variety of different expression constructs, such as expression cassettes and vectors suitable for transformation of plant cells can be prepared. A DNA sequence coding for a polypeptide described in the present invention, e.g., a cDNA sequence encoding BRI1, or a BRI1 domain, can be combined with cis-acting (promoter and enhancer) transcriptional regulatory sequences to direct the timing, tissue type and levels of transcription in the intended tissues of the transformed plant. Translational control elements can also be used.

The invention provides a nucleic acid encoding a BRI1 polypeptide operably linked to a promoter which is capable of driving the transcription of the coding sequence in plants. The promoter can be, e.g., derived from plant or viral sources. The promoter can be, e.g., constitutively active, inducible, or tissue specific. In construction of recombinant expression cassettes, vectors, transgenics, of the invention, different promoters can be chosen and employed to differentially direct gene expression, e.g., in some or all tissues of a plant.

Further provided are methods of generating transgenic plants that express recombinant BRI1 (or other desired protein). Appropriate expression cassettes can be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using biolistics, e.g., DNA particle bombardment.

Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Biolistic transformation techniques are described in Klein et al. Nature 327:70-73 (1987).

Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983) and Gene Transfer to Plants, Potrykus, ed. (Springer-Verlag, Berlin 1995).

Transformed plant cells which are derived by any of the above transformation techniques can be cultured to regenerate a whole plant which possesses a desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al., Ann. Rev, of Plant Phys. 38:467-486 (1987).

The above techniques can be used to produce transgenic plants in any plant species, including species from the genera Anacardium, Arachis, Asparagus, Atropa, Avena, Brassica, Chlamydomonas, Chlorella, Citrus, Citrullus, Capsicum, Carthamus, Cocos, Coffea, Cucumis, Cucurbita, Cyrtomium, Daucus, Elaeis, Fragaria, Glycine, Gossypium, Helianthus, Heterocallis, Hordeum, Hyoscyamus, Lactuca, Laminaria, Linum, Lolium, Lupinus, Lycopersicon, Macrocystis, Malus, Manihot, Majorana, Medicago, Nereocystis, Nicotiana, Olea, Oryza, Osmunda, Panieum, Pannesetum, Persea, Phaseolus, Pistachia, Pisum, Pyrus, Polypodium, Prunus, Pteridium, Raphanus, Ricinus, Secale, Senecio, Sinapis, Solanum, Sorghum, Theobromus, Trigonella, Triticum, Vicia, Vitis, Vigna, and Zea.

### IV. Methods of Identifying a BRI1 Modulator

A. BRI1 Activity Assays

BRI1 activities include binding to brassinosteroids (e.g. brassinolide and compounds disclosed in Back & Pharis (2003) J. Plant Growth Regul. 22:350), BAK1, and BAK1-like proteins. BRI1 modulators can also bind BRI1, e.g., to interfere with ligand or coreceptor binding (antagonist), or to mimic or improve ligand or coreceptor binding (agonist).

The binding affinity of a compound, e.g., a candidate BRI1 modulator, can be defined in terms of the comparative dissociation constants (Kd) of the compound for target (e.g., BRI1), as compared to the dissociation constant with respect to the compound and other materials in the environment or unrelated molecules in general. Typically, the Kd for the compound with respect to the unrelated material will be at least 2-fold, 3-fold, 4-fold, 5-fold, 10-fold, 20-fold, 50-fold, 100-fold, 200-fold or higher than Kd with respect to the target.

The desired affinity for an compound, e.g., high (pM to low nM), medium (low nM to 100 nM), or low (about 100 nM or higher), can differ depending upon the BRI1 binding site and the targeted activity. Compounds having different affinities can be used for different applications.

A compound will typically bind with a Kd of less than about 1000 nM, e.g., less than 250, 100, 50, 20 or lower nM. In some embodiments, the Kd of the compound is less than 15, 10, 5, or 1 nM. The value of the dissociation constant (Kd) can be determined by well-known methods, and can be computed even for complex mixtures by methods as disclosed, e.g., in Caceci et al., Byte (1984) 9:340-362; and as reviewed in Ernst et al. Determination of Equilibrium Dissociation Constants, Therapeutic Monoclonal Antibodies (Wiley & Sons ed. 2009).

The Kd, Kon, and Koff can also be determined using surface plasmon resonance (SPR), e.g., as measured by using a Biacore T100 system. SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given frequency is shined on the surface, the changes to the optical reflectivity of the gold indicate binding, and the kinetics of binding.

Binding affinity can also be determined by anchoring a biotinylated interactant to a streptaviden (SA) sensor chip. The other interactant is then contacted with the chip and detected, e.g., as described in Abdessamad et al. (2002) Nuc. Acids Res. 30:e45.

BRI1 activities also include BAK1 phosphorylation and initiation of the brassinosteroid signaling pathway. Assays for detection of BRI1 signal transduction are described, e.g., in Wang et al. (2006) Cell Res. 16:427.

Methods for detecting increased plant mass or increased size of vegetative structure can include the steps of exposing a BRI1 expressing plant with a BRI1 agonist and detecting an increase in the amount of plant mass and/or size of vegetative structures (e.g., stems, leaves) as compared to a standard control. An appropriate standard control can be selected by one of skill in the art, e.g., a plant that does not express BRI1, a plant that is not exposed to a BRI1 agonist, or a plant that is exposed to a BRI1 antagonist.

BRI1 activity can be measured using a leaf lamina inclination assay (Baron et al. (1998) Phytochemistry 49:1849; Back & Pharis (2003) J. Plant Growth RegulI 22:350). In the absence of brassinosteroid signaling, the leaf lamina is nearly vertical, e.g., 160-170 degrees, while strong brassinosteroid signaling results in a leaf lamina angle of about 60 degrees.

In some embodiments, the invention provides methods of identifying a BRI1 modulator comprising contacting a candidate compound and BRI1, and detecting BRI1 activity, wherein a change in BRI1 activity in the presence of the candidate compared to a standard control indicates that the candidate compound is a BRI1 modulator. In some embodiments, the BRI1 is expressed in a plant, and the contacting step involves contacting the candidate compound with the plant. In some embodiments, the BRI1 modulator is a BRI1 inhibitor, and in some embodiments, the BRI1 modulator is a BRI1 agonist.

In some embodiments, the standard control lacks the candidate compound. In some embodiments, e.g., for determining whether the candidate compound is a BRI1 agonist, the standard control is brassinolide, or another known BRI1 agonist. In some embodiments, e.g., for determining whether the candidate compound is a BRI1 antagonist, the method further includes a step of exposing the BRI1 to an agonist, and determining the ability of the candidate compound to interfere with BRI1 signaling.

The presently provided structural data allows one of skill to more accurately design and/or identify potential BRI1 modulators, e.g., based on known modulators, the structural elements of the ligand-binding site, or the structural elements of the co-receptor interaction site.

B. BRI1 Variants and Modulators

Provided herein are BRI1 variants and modulators that can be used for comparison, e.g., as controls, in the screening methods described herein. For example, the activity of a candidate BRI1 modulator can be compared to that of a known BRI1 modulator. The activity of a candidate BRI1 modulator can also be compared to the activity of a BRI1 variant, e.g., a gain-of-function mutant (Haliday et al. 2006 Plant J.) or loss-of-function mutant (Grove et al. 1979 Nature; Nam & Li 2002 Cell).

BRI1 variants include loss-of-function mutants 102 (Thr75011e), 6 (Gly644Asp), and gain-of-function mutation sud1 (Gly643Glu) (see, e.g., Noguchi et al. (1999) Plant Physiol 121:743; Dievart et al. (2006) Funct Plant Biol. 33:723; Friedrichsen et al. (2000) Plant Physiol 123:1247). For example, a candidate modulator that causes a dwarf phenotype similar to bri1-102 compared to an untreated BRI1 wild-type plant can be considered a BRI1 antagonist or inhibitor. A candidate modulator that causes a larger (increased biomass, increase vegetative structure size) phenotype, similar to sud1, compared to an untreated BRI1 wild-type plant can be considered a BRI agonist or activator.

Compounds with BRI1 agonist activity include, but are not limited to, the steroidal and non-steroidal brassinolide-like compounds disclosed in Back & Pharis (2003) J. Plant Growth Regul 22:350; and the brassinolide B-ring analogs 7-azabrassinolide, 7-thiabrassinolide, 6-deoxybrassinolide, B-homocastasterone, 6-methylidene-castasterone and 6-methylidene-B-homocastasterone (Baron et al. (1998) Phytochemistry 49:1849). The activity of a candidate BRI1 modulator can be compared with these BRI1 agonists, as well as brassinolide itself, to determine if the candidate modulator is also an agonist. BRI1 antagonists include BKI, which can be used for comparison, e.g., to determine if a candidate modulator is an antagonist.

C. Rational Design of BRI1 Modulators

Hormones, hormone mimetics, and other modulating compounds with BRI1 regulating activity can be identified using structure coordinates of the BRI1 ectodomain, BRI1 island domain, or other BRI1 domains, as disclosed herein. Such methods of screening can comprise: (a) generating structure coordinates of a three-dimensional structure of a test substance; and (b) superimposing the structure coordinates of (a) onto all or some of the structure coordinates of BRI1 in the same coordinate system so as to evaluate their state of fitting. Specifically, such a method involves fitting the structure coordinates of BRI1 to structure coordinates representing a three-dimensional structure of any test substance on a computer, expressing their state of fitting numerically using, for example, empirical scoring functions as indices, and then evaluating the binding ability of the test substance to BRI1.

The structure coordinates of BRI1 are used, the shape of BRI1 binding site or interaction site is assigned, and then a compound that can bind to the site can be subjected to computer screening using commercial package software such as DOCK (Ewing et al., J. COMP. AIDED MOL. DES. 15:411-428 (2001)), AutoDock (Morris et al., J. COMPUTATIONAL CHEM. 19:1639-1662 (1998)), Ludi, or LigandFit. For example, amino acid residues and domains in BRI1 that can interact with the natural brassinolide ligand are shown, e.g., in Table 2 and FIG. 9. Thus, it becomes possible to conduct computer screening using such sites as an aid.

The step of superimposing structure coordinates of a test substance onto all or some of the structure coordinates of BRI1 in the same coordinate system so as to evaluate their state of fitting can also be carried out with the above commercial software. Any appropriate modeling software can be used, as long as it makes a simulation of the docking procedure of a ligand or other modulator to a protein possible on a computer. For example, software programs such as DOCK, FlexX (Tripos, Inc.), LigandFit (Accelrys Inc.), or Ludi (Accelrys Inc.) can be used.

In some embodiments, an initial step is positioning of a virtual spherical body referred to as a sphere, using a SPHGEN program, near a position to which a candidate BRI1 modulator (agonist or antagonist) is likely to bind. This sphere functions as an anchor for docking of the modulator. In addition, sites at which spheres are generated can be limited to specific pockets or specific clefts, or spheres can be generated at a plurality of sites.

Next, grids are generated at a portion and the periphery of the desired BRI1 position using a GRID program, so as to express an electronic and steric environment for receptor residues within an assigned range as a scalar value on each grid. In addition, the force field of AMBER (Pearlman, et al., COMP. PHYS. COMMUN. 91:1-41 (1995)) or the like is utilized to calculate each grid value. Furthermore, depending on the shape, adjustment can also be made by altering grid information so as to express docking of a compound in a more realistic form.

Next, a search can be conducted on a compound database. Using the DOCK program, a compound that is takes a three-dimensional conformation so as not to repel steric elements or electronic elements on the grids is searched for. The three dimensional conformation of the docked compound is optimized by a conformation-generating function integrated in the DOCK program. Whether or not appropriate docking is finally conducted can be further determined based on empirical judgment, e.g., using scores at the time of docking, visual observation, and in situ screening. In this manner, a series of selected compound groups judged to be able to appropriately conduct docking can be considered as substances likely to modulate BRI1 activity (agonist or antagonist) at a certain probability.

The above method promotes more efficient, rational development of BRI1 modulators. Specifically, predicting the arrangement of structure coordinates that fit the properties and shapes of the interaction sites of the BRI1-ligand complex, or the BRI1-BAK1 complex, and the selection by calculation of a compound having a structure capable of agreeing with the putative structure coordinates, make it possible to efficiently select an activity-controlling substance specific to BRI1 from among many compounds.

Likely modulator compounds obtained from the modeling methods can then be validated using any of the screening methods described above, e.g., by contacting the likely modulator compound with a plant expressing BRI1, and determining the effect of the compound on the plant.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

### V. Examples

### A. Methods

The entire BRI1 ectodomain was produced as a StrepII-9xHis fusion protein by secreted expression in baculovirus-infected Trichoplusia ni cells. The protein was harvested 4 d post-infection by ultrafiltration and purified by sequential Co²⁺ and Strep affinity chromatography, and by gel filtration. BRI1 was concentrated to 15 mg/ml and crystallized by vapor diffusion using a reservoir solution containing 14% PEG 4,000, 0.2 M (NH₄)₂SO₄, 0.1 M citric acid (pH 4.0). A complex with the plant steroid brassinolide was obtained by co-crystallization. Diffraction data to 2.5 Å resolution were collected on a rotating anode X-ray generator and at beam-line 8.2.1 of the Advanced Light Source (ALS), Berkeley. The structure was solved using the SIRAS method. Data and refinement statistics are summarized in Table 1.

Protein expression and purification. A synthetic gene comprising the entire BRI1 ectodomain (residues 29-788) and codon optimized for expression in Trichoplusia ni was synthesized by Geneart (Regensburg, Germany). The gene was cloned into a modified pBAC-6 transfer vector (Novagen), providing a glycoprotein 64 signal peptide and a C-terminal TEV (tobacco etch virus protease) cleavable Strep-9xHis tandem affinity tag. Recombinant baculoviruses were generated by co-transfecting the transfer vector with linearized baculovirus DNA (ProFold-ER1, AB vector, San Diego, USA) and amplified in Sf9 cells. The fusion protein was expressed in Hi5 cells using a multiplicity of infection of 5, and harvested from the medium 4 days post infection by tangential flow filtration using a 30 kDa MWCO (molecular weight cut-off) filter membrane (GE Healthcare). BRI1 was purified by sequential Co²⁺ (His select gel, Sigma) and Strep (Strep-Tactin Superflow high-capacity, IBA, Gottingen, Germany) affinity chromatography. Next, the tandem affinity tag was removed by incubating purified BRI1 with recombinant TEV protease in 1:100 molar ratio. The cleaved tag and the protease were separated from BRI1 by size exclusion chromatography on a Superdex 200 HR10/30 column (GE Healthcare) equilibrated in 20 mM Hepes pH 7.5, 100 mM NaCl, 1 mM EDTA). Monomeric peak fractions were concentrated to ˜15 mg/mL and snap frozen in liquid nitrogen. About 50-80 μg of purified BRI1 could be obtained from 1 litre of insect cell culture.

Crystallization and data collection. Initial crystals of BRI1 appeared in 18% PEG 4,000, 0.8 M KCl using the counter diffusion method. Diffraction quality crystals of about 300×80×600 um could be grown after multiple rounds of microseeding at room-temperature by vapor diffusion in hanging drops composed of 1.25 μL of protein solution (15 mg/mL) and 1.25 μL of crystallization buffer (14% PEG 4,000, 0.2 M (NH₄)₂SO₄, 0.1 M citric acid pH 4.0) suspended above 1.0 mL of the mother liquor as the reservoir solution. For structure solution crystals were stabilized, derivatized and cryo-protected by serial transfer into 16% PEG 4,000, 1.7 M Na malonate pH (4.0) and 0.5 M NaI, and cryo-cooled in liquid nitrogen. Single-wavelength anomalous diffraction (SAD) data to 2.9 Å resolution were collected on a Rigaku MicroMax rotating anode equipped with a copper filament, osmic mirrors and an R-AXIS IV++ detector. Native crystals were transferred to a cryo-protective solution containing 16% PEG 4,000 and 1.7 M Na malonate (pH 4.0) and flash-cooled in liquid nitrogen. An isomorphous native dataset to 2.5 Å was collected at beam-line 8.2.1 of the Advanced Light Source (ALS), Berkeley. The hormone-bound structure was obtained by dissolving brassinolide (Chemiclones Inc., Waterloo, Canada) to a concentration of 1 mM in 100% DMSO. This stock solution was diluted to a final concentration of about 50 μM in protein storage buffer (20 mM Hepes pH 7.5, 100 mM NaCl, 1 mM EDTA). Purified BRI1 protein was added to a final concentration of about 12.5 μM (1.5 mg/mL) and the mixture was incubated at room-temperature for 16 h. Next, the complex was re-concentrated to 18 mg/mL, and immediately used for crystallization. Crystals appeared under similar conditions as established for the unbound form and diffracted again to about 2.5 Å. Data processing and scaling was done with XDS (Kabsch, J. Appl. Crystallogr. 26, 795-800 (1993)) (version: May 2010) (Table 1).

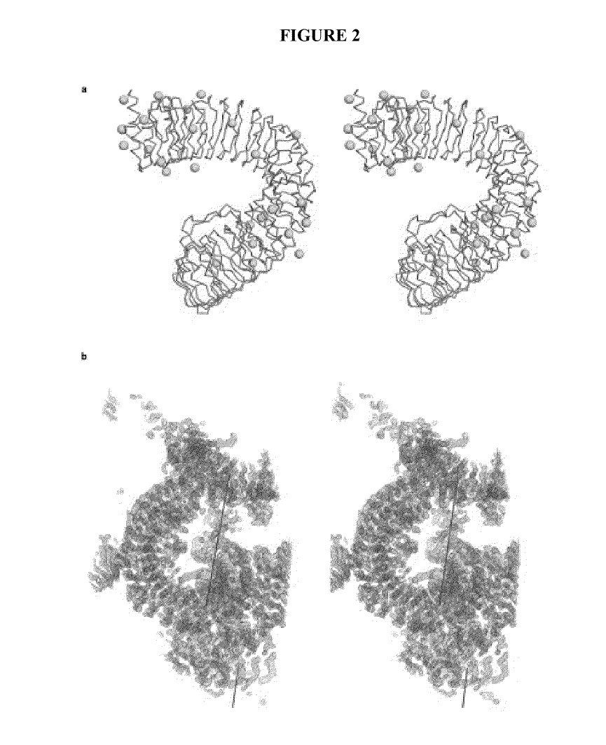

Structure Solution and Refinement. The program XPREP (Bruker AXS) was used to scale native and derivative data for SIRAS (single isomorphous replacement with anomalous scattering) analysis. Using data between 30-3.7 Å, SHELXD (Sheldrick, Crystallogr. 64, 112-122 (2008)) located 52 iodine sites (CC All/Weak 42.50/19.82). 16 consistent sites were input into the program SHARP (Bricogne et al., Acta Crystallogr. D Biol. Crystallogr. 59, 2023-2030 (2003)) for phasing and identification of 10 additional sites at 2.9 Å resolution (FIG. 2a). Refined heavy atom sites and phases were input into phenix.resolve (Terwilliger et al., Acta Crystallogr. D Biol. Crystallogr. 64, 61-69 (2008)) for density modification and phase extension to 2.5 Å (final FOM was 0.55). The resulting electron density map was readily interpretable (FIG. 2b), and the structure was completed in alternating cycles of model building in COOT (Emsley & Cowtan, Acta Crystallogr. DBiol. Crystallogr. 60, 2126-2132 (2004)) and restrained TLS refinement in phenix.refine (Afonine et al., CCP4 Newsl. contribution 8 (2005)). Refinement statistics are summarized in Table 1. The crystals contain one BRI1 monomer per asymmetric unit with a solvent content of ˜60%. The final models comprise residues 29-771, with the C-termini (residues 772-788) being completely disordered. The structure contains 25 LRRs as initially proposed (Nadeau, J. A. & Sack, F. D., Science 296, 1697-1700 (2002)), and not 24 LRRs as concluded from later modeling studies (Vert et al., Annu. Rev. Cell Dev. Biol 21, 177-201 (2005)). Loop residues 590, 637 and 638 in the island domain appear disordered in the unliganded structure. Amino acids whose side-chains could not be modeled with confidence were truncated to alanine (2% of all residues). Analysis with Molprobity (Davis et al., Nucleic Acids Res. 35, W375-383 (2007)) suggested that both refined models have excellent stereochemistry, with the free form having 93.3% of all residues in the favored region of the Ramachandran plot, and no outliers (Molprobity score is 2.2 corresponding to the 90^{th} percentile for structures (N=6,681) at 2.52 Å f 0.25 Å resolution). The brassinolide complex structure has 92.7% of all residues in the favored region of the Ramachandran plot and no outliers (Molprobity score is 2.3 corresponding to the 86^{th} percentile for structures (N=6,632) at 2.54 Å f 0.25 Å resolution). Structural visualization was done with POVScript+ (Fenn et al., J. Appl. Crystallogr. 36, 944-947 (2003)) and POV-Ray (available at the website at povray.org).

Size-exclusion chromatography was performed using a Superdex 200 HR 10/30 column (GE Healthcare) pre-equilibrated in 25 mM citric acid/sodium citrate buffer (pH 4.5), 100 mM NaCl. 100 μL of sample (5 mg/mL) was loaded onto the column and elution at 0.6 mL/min was monitored by ultraviolet absorbance at 280 nm. Incubation with brassinolide was performed as described in the crystallization section.

Homology Modeling of the AtBAK1 ectodomain (residues 27-227; Uniprot accession Q94F62) was performed with the program MODELLER using the BRI1 and PGIP structures as template. Structure-based sequence alignments were done using T-COFFEE (Notredame et al., J. Mol. Biol. 302, 205-217 (2000)). BRI1 and BAK1 share −35%, PGIP and BAK1 share −31% sequence identity, with the LRR and N-cap consensus sequences being highly conserved.

### B. Example 1

### Overall Structure of the BRI1 Ectodomain

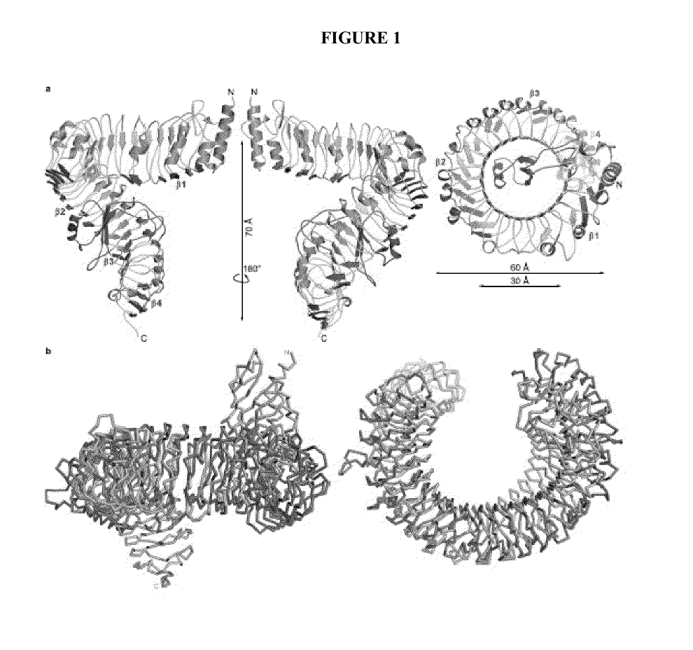

The BRI1 ectodomain (residues 29-788) was expressed in baculovirus-infected insect cells and the secreted protein was purified by tandem-affinity and size-exclusion chromatography. Crystals diffracted to 2.5 Å resolution, and the structure was solved by single isomorphous replacement (see Table 1 and FIG. 1). BRI1 does not adopt the anticipated TLR-horseshoe structure but forms a right-handed superhelix composed of 25 LRRs (FIG. 1a). The helix completes slightly more than one full turn, with a rise of ˜70 Å. The concave surface, that determines the curvature of the solenoid (Bella et al., Cell. Mol. Life. Sci. 65, 2307-2333 (2008)), is mainly formed by α- and 3₁₀ helices (green in FIG. 1a) that cause inner and outer diameters of ˜30 and ˜60 Å, respectively (FIG. 1a). The overall curvature of BRI is similar to TLR3 (Choe et al., Science 309, 581-585 (2005)) (FIG. 1b), but, while the TLR3 ectodomain is essentially flat, BRI1 is highly twisted (FIG. 1b).

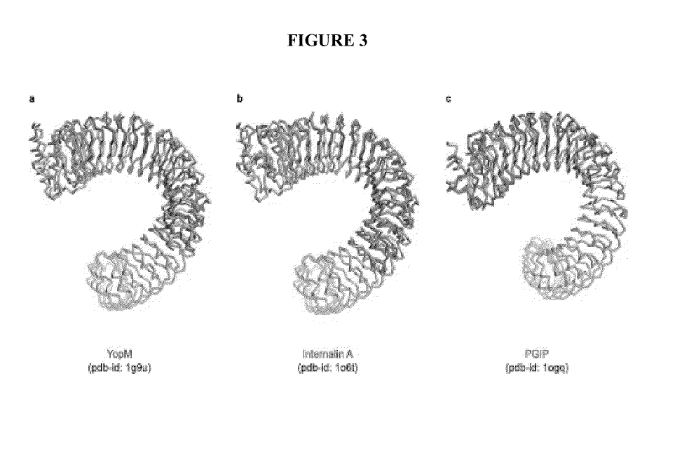

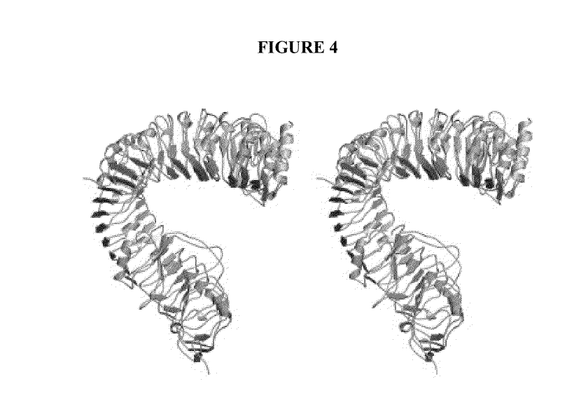

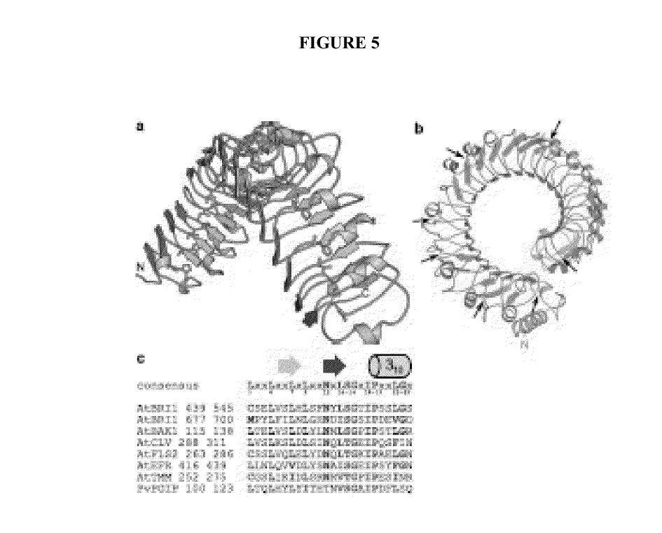

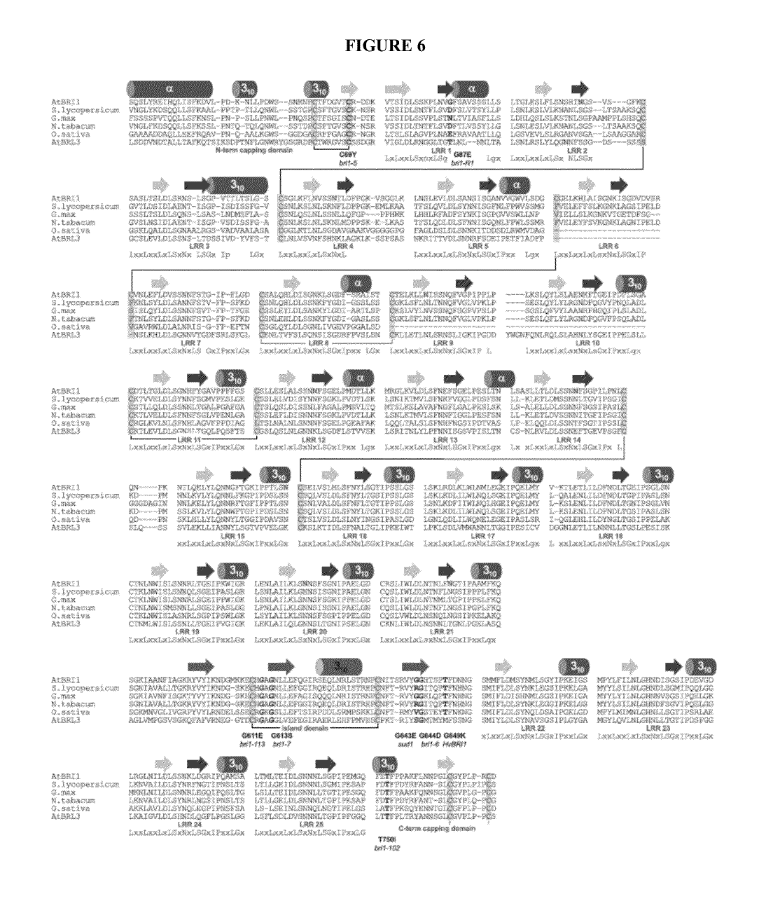

Such twisted assemblies of LRRs have been observed previously with bacterial effector (Evdokimov et al., J. Mol. Biol. 312, 807-821 (2001)) and adhesion proteins (Schubert et al., Cell 111, 825-836 (2002)), and with the plant defense protein PGIP (FIG. 3a). The twist of PGIP's LRR domain is caused by a non-canonical, second β-sheet that is oriented perpendicular to the central β-sheet and forms the inner surface of the solenoid. Additional β-sheets are also present in our structure (blue in FIG. 1a, FIG. 3), but in the case of the much larger BRI1 ectodomain result in a superhelical assembly (FIG. 1a). The second β-strand in PGIP and in BRI1 is followed by an Ile-Pro spine that runs along the outer surface of the helix and provides packing interactions between consecutive LRRs (FIG. 4a). Both structural features are directly linked to the Lt/sGxIP consensus sequence that is the defining fingerprint for the plant-specific LRR subfamily (Kajava, J. Mol. Biol. 277, 519-527 (1998)) (FIG. 5c, FIG. 6 and Table 2). Because this consensus sequence is found in other plant RKs, these receptors may also harbor twisted LRR domains (FIG. 5c), making BRI1 the primary template for the study of diverse signaling pathways in plants.

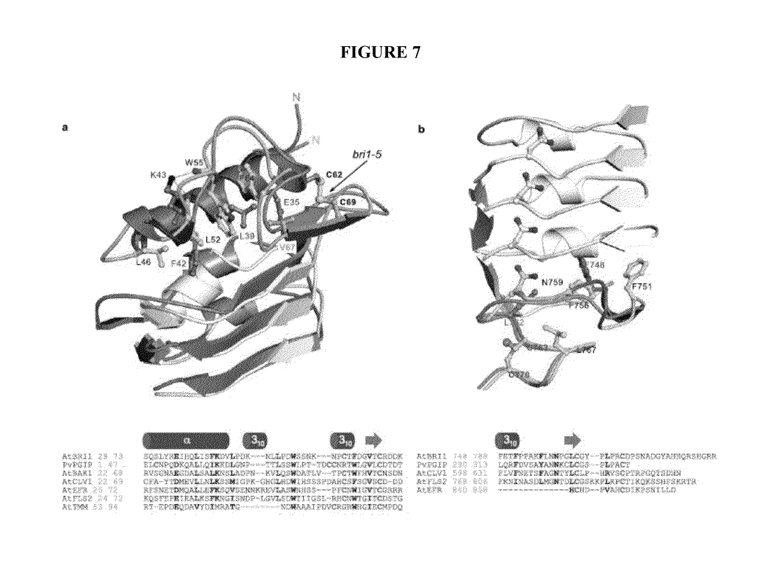

The N- and C-terminal flanking regions that cap the hydrophobic core of the BRI1 solenoid are similar to caps previously described for PGIP (FIG. 7). Not are only these caps stabilized by disulfide bridges, but cysteines in position 1 and 24 of the 24-residue LRR motif result in 5 additional disulfide bonds that link consecutive LRR segments in the N-terminal half of the BRI1 ectodomain (FIG. 5b, FIG. 6, and Table 2).

TABLE 1

| | unbound (NaI soak) | unbound (native) | brassinolide-complex |
|---|---|---|---|
| Data collection | | | |
| Space group | C2 | C2 | C2 |
| Cell dimensions | | | |
| a, b, c (Å) | 175.04, 67.53, 119.83 | 175.09, 67.25, 119.05 | 175.11, 67.21, 119.21 |
| β angle (°) | 121.06 | 121.55 | 121.41 |
| Wavelength (Å) | 1.5418 | 0.9998 | 1.5418 |
| Resolution (Å) | 29.28-2.90 | 31.00-2.52 | 24.64-2.54 |
| Highest shell (Å) | 2.97-2.90 | 2.68-2.52 | 2.69-2.54 |
| No. unique reflections* | 26.445 (1.625) | 39.686 (6.145) | 38.900 (5.904) |
| R [symbol] (%) [symbol] | 8.0 (47.8) | 5.5 (58.3) | 6.0 (48.2) |
| //σ/ | 22.2 (4.1) | 17.6 (2.1) | 14.9 (2.3) |
| Completeness (%) | 97.7 (81.4) | 98.8 (95.3) | 98.6 (93.8) |
| Multiplicity | 14.2 (10.5) | 3.7 (3.5) | 4.1 (3.5) |
| Refinement | | | |
| Resolution (Å) | | 31.00-2.52 | 24.64-2.54 |
| Highest shell (Å) | | 2.61-2.52 | 2.63-2.54 |
| No. reflections | | 39.686 (3.575) | 38.849 (3.537) |
| R [symbol] | | 0.185 (0.321) | 0.184 (0.263) |
| R [symbol] | | 0.236 (0.409) | 0.240 (0.332) |
| No. atoms | | | |
| Protein/glycan | | 5.544/170 | 5.558/192 |
| Water/brassinolide | | 129 | 114/34 |
| B-factors (Å [symbol] ) | | | |
| Wilson B | | 55.0 | 51.8 |
| Protein/glycan | | 62.5/92.9 | 64.5/89.3 |
| Water/brassinolide | | 53.0 | 51.4/47.8 |
| R.m.s. deviations | | | |
| bond length (Å) | | 0.006 | 0.006 |
| bond angles (°) | | 1.02 | 1.05 |

*Numbers in parentheses provide statistics for the highest resolution shell [symbol]
As defined in XDS [symbol] [symbol]
As defined in phenix.refine [symbol] [symbol]
indicates data missing or illegible when filed

TABLE 2

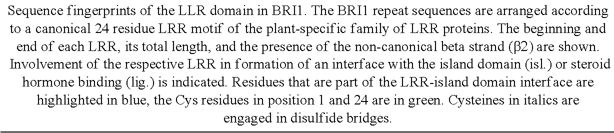

| | | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | | No. | | isl./ |
|---|---|---|---|---|---|---|---|---|---|---|---|---|---|---|---|---|---|---|---|---|---|---|---|---|---|---|---|---|---|
| LRR | bcg | L | X | X | L | X | X | L | X | L | S | X | N | X | L | S | G | X | I | P | X | X | L | G | X | cnd | res. | β2 | lig. |
| 1 | 74 | | | | V | T | S | I | D | L | S | S | K | P | L | N | V | G | P | S | A | V | S | SL | LS | 97 | 23 | + | / |
| 2 | 98 | L | T | G | L | E | S | L | F | L | S | N | S | H | I | N | G | S | V | S | G | F | K | C | | 120 | 23 | + | / |
| 3 | 123 | S | A | S | L | T | S | L | D | L | C | R | N | S | L | S | G | P | V | T | T | L | T | SL | GS | 146 | 26 | + | / |
| 4 | 147 | C | S | G | L | K | F | L | N | V | S | S | N | T | L | D | P | P | G | K | V | S | G | G | LK | 171 | 25 | + | / |
| 5 | 172 | L | N | S | L | E | V | L | D | L | S | A | N | S | I | S | G | A | N | V | V | G | WV | LS | DG | 198 | 27 | + | / |
| 6 | 199 | C | G | E | L | K | H | L | A | I | S | G | N | K | I | S | G | D | | | | | V | S | R | 220 | 20 | + | / |
| 7 | 221 | C | V | N | L | E | F | L | E | V | S | S | N | N | F | S | T | G | I | P | F | | L | G | D | 243 | 23 | | / |
| 8 | 244 | C | S | A | L | Q | H | L | D | I | S | G | N | K | L | S | G | D | P | S | R | A | I | S | T | 267 | 24 | | / |
| 9 | 268 | C | T | E | L | K | L | L | N | I | S | S | N | O | F | V | G | P | I | P | P | | L | P | | 289 | 22 | + | / |
| 10 | 290 | L | K | S | L | O | Y | L | S | L | A | E | N | K | F | T | G | E | I | P | D | F | L | SG | A | 314 | 25 | + | / |
| 11 | 315 | C | D | T | L | T | G | L | D | L | S | G | N | H | F | Y | G | A | V | P | P | F | F | G | S | 338 | 24 | + | / |
| 12 | 339 | C | S | L | L | E | S | L | A | L | S | S | N | N | F | S | G | E | L | P | M | DT | L | L | K | 363 | 25 | + | / |
| 13 | 364 | M | K | G | L | K | V | L | D | L | S | F | N | E | P | S | G | E | L | P | E | S | L | T | N | 387 | 24 | + | +/ |
| 14 | 388 | L | S | AS | L | L | T | L | D | L | S | S | N | N | F | S | G | P | I | L | P | N | L | C | | 411 | 24 | + | +/ |
| 15 | 416 | | N | T | L | O | E | L | Y | L | O | N | N | G | F | T | G | K | I | P | P | T | L | S | N | 438 | 23 | + | +/ |
| 16 | 439 | C | S | E | L | V | S | L | H | L | S | F | N | Y | L | S | G | T | I | P | S | SS | L | G | S | 462 | 25 | + | +/ |
| 17 | 463 | L | S | K | L | R | D | L | K | L | W | L | N | M | L | E | G | E | I | P | Q | E | L | M | Y | 486 | 24 | + | +/ |
| 18 | 487 | V | K | T | L | E | T | L | I | L | D | F | N | E | L | T | G | E | I | P | S | G | L | S | N | 510 | 24 | + | +/ |
| 19 | 511 | C | T | N | L | N | W | I | S | L | S | N | N | R | L | T | G | E | I | P | K | W | I | G | R | 534 | 24 | + | +/ |
| 20 | 535 | L | E | N | L | A | I | L | K | L | S | N | N | S | F | S | G | N | I | P | A | E | L | G | D | 558 | 24 | + | +/ |
| 21 | 559 | C | R | S | L | I | W | L | D | L | N | T | N | L | F | N | G | T | I | P | A | A | MF | K | Q | 583 | 25 | + | +/+ |
| 22 | 655 | | | S | M | M | F | L | D | M | S | Y | N | M | L | S | G | Y | I | P | K | E | I | G | S | 676 | 22 | | +/+ |
| 23 | 677 | M | P | Y | L | F | I | L | N | L | G | H | N | D | I | S | G | S | I | P | D | E | V | G | D | 700 | 24 | + | +/+ |
| 24 | 701 | L | R | G | L | N | I | L | D | L | S | S | N | K | L | D | G | R | I | P | Q | A | M | S | A | 724 | 24 | + | +/+ |
| 25 | 725 | L | T | M | L | T | E | I | D | L | S | N | N | N | L | S | G | R | I | P | Q | A | M | G | Q | 747 | 24 | + | +/+ |

### C. Example 2

### The Island Domain

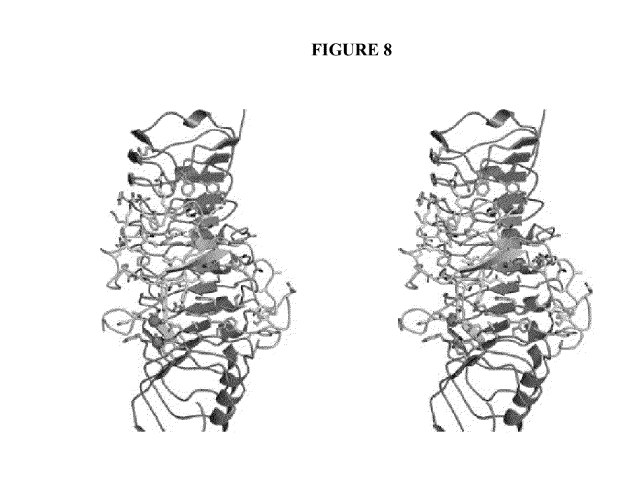

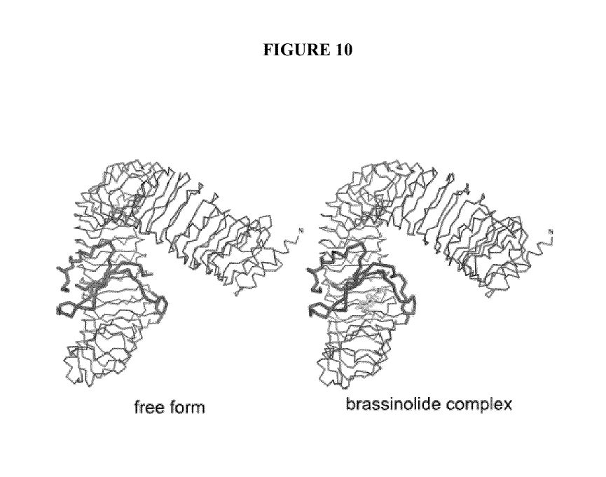

The island domain in BRI1 corresponds to a large insertion in the regular repeat-structure between LRRs 21 and 22 (residues 584-654) (FIG. 1a). The resulting ˜70 residue segment forms a small domain that folds back into the interior of the superhelix, where it makes extensive polar and hydrophobic interactions with LRRs 13-25 (FIG. 1a, FIG. 8, and Table 2). The domain fold is characterized by an anti-parallel O-sheet, which is sandwiched between the LRR core and a 3₁₀ helix and stabilized by a disulfide bridge (FIG. 9a, FIG. 6). The loss-of-function alleles bri1-9 (Ser662Phe, weak) (Noguchi et al., Plant Physiol. 121, 743-752 (1999)) and bri1-113 (Gly611Glu, strong) (Li & Chory Cell 90, 929-938 (1997)) map to this island domain—LRR interface (FIG. 8), and likely interfere with folding of the island domain (En et al., Mol. Cell. 26, 821-830 (2007)). Two long loop segments that connect the island domain to the LRR core appear partially disordered in the unliganded receptor (FIG. 10). The insertion of a folded domain into the LRR repeat has not been observed in other LRR receptor structures and is likely an adaptation to the challenge of sensing a small steroid ligand (compared, for example, to recognizing larger ligands, such as proteins, nucleic acids, or lipids).

### D. Example 3

### Brassinolide—BRI1 complex

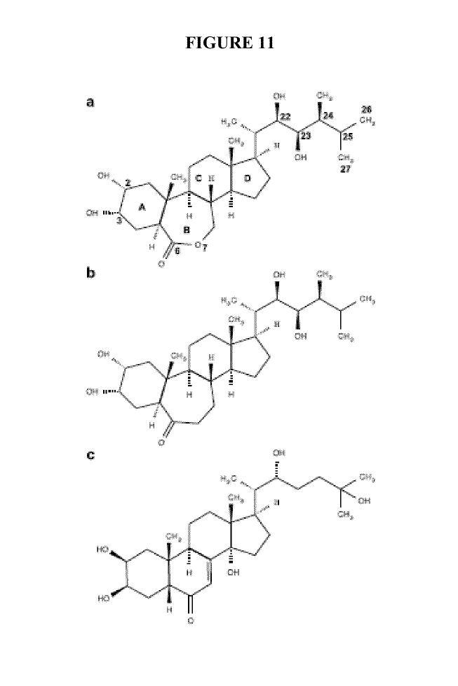

We next solved a 2.5 Å co-crystal structure with brassinolide, a potent brassinosteroid that binds BRI1 with nanomolar affinity. Difference density accounting for one molecule of brassinolide per BRI1 monomer was found in close proximity to the island domain (FIG. 9a-c), which was previously implicated in steroid binding. The structure reveals that the LRR superhelix and the island domain both extensively contribute to formation of the hormone binding site. The A-D rings of the steroid bind to a hydrophobic surface which is provided by LRRs 23-25 and that maps to the inner side of the BRI1 superhelix (FIG. 9b,d, FIG. 11). The alkyl chain of the hormone fits into a small pocket formed by residues originating from LRRs 21 and 22 (Ile563, Trp564, Met657, Phe658) and from two long loops connecting the island domain with the LRR core (FIG. 9d). The hydrophobic nature and restricted size of this pocket now explain why steroid ligands with bulkier or charged alkyl side chains, such as the arthropod steroid ecdysone (FIG. 11), cannot be recognized by BRI1. A few polar interactions with the second brassinolide diol moiety (FIG. 9d) are established with Tyr597 and main chain atoms from His645 and Ser647 in the island domain, and are mediated by water molecules (FIG. 9d). Mutation of the neighboring Gly644 to Asp may interfere with this hydrogen bonding network, and explain why this mutation greatly reduces the binding activity of the receptor and causes the loss-of-function phenotype bri1-6 (FIG. 9d). No polar contacts are observed with the seven-membered B-ring lactone (FIG. 9d), consistent with B-ring modifications as found in e.g. castasterone (FIG. 11) being tolerated by BRI1.

The steroid-complex reveals a hormone-binding site that involves a much larger portion of the LRR domain than expected. Major interactions between the steroid and the BRI1 LRR domain originate from the very C-terminal LRRs 23-25, which brings the hormone in close proximity to the membrane (FIG. 9a,d). Importantly, while there is a significant hormone-receptor interface (550 Å²) for such a small molecule ligand, large parts of the steroid are exposed to the solvent, including the 2α,3α-diol moiety in brassinolide that is important for biological activity (Back & Pharis, J. Plant Growth Regul. 22, 350-361 (2003)). The structure indicates that protein-protein interactions are involved in the recognition of the steroid ligand, with the hormone itself providing a docking platform. Steroid binding induces a conformational rearrangement and fixing of the island domain, which can then become fully ordered and competent to participate in the interactions critical for receptor activation (see below) (FIG. 10).

### E. Example 4

### Glycosylation Sites

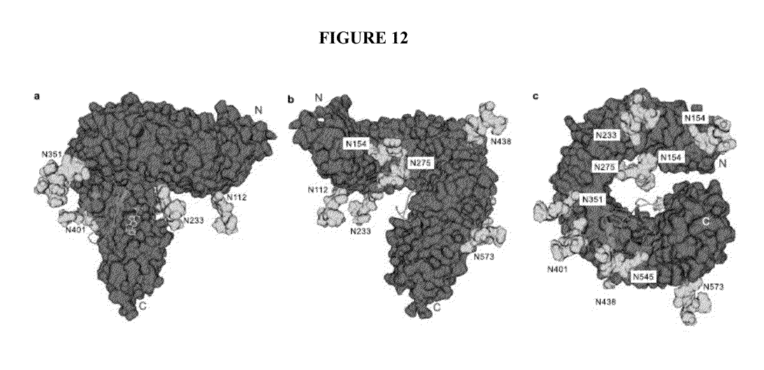

We observed electron density for nine N-glycosylation sites (Asn¹¹², Asn¹⁵⁴, Asn²³³, Asn²⁷⁵, Asn³⁵¹, Asn⁴⁰¹, Asn⁴³⁸, Asn⁵⁴⁵, Asn⁵⁷⁵). Particularly well ordered glycans are found at Asn¹⁵⁴ and Asn²⁷⁵, which map to the interior of the superhelix and may have a role in structural stabilisation (FIG. 12a-c). Glycans on the inner surface of the LRR domain are also found in TLR3 (Choe et al., Science 309, 581-585 (2005)). A well-ordered glycan is positioned at Asn⁵⁴⁵, where it establishes contacts with the distal side of the island domain (FIG. 12c). Overall, the carbohydrates mask large surface areas of the N-terminal half of BRI1, as well as the inner face of the superhelix, but are occluded from the very C-terminal part of BRI1 that harbours the steroid binding site (FIG. 12a-c).

### F. Example 5

### The Interaction Surface

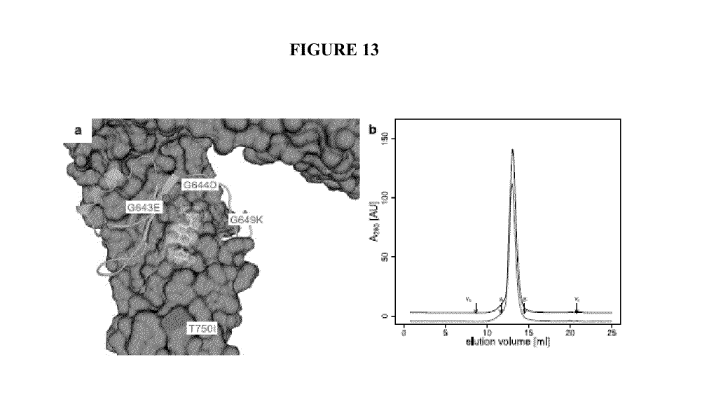

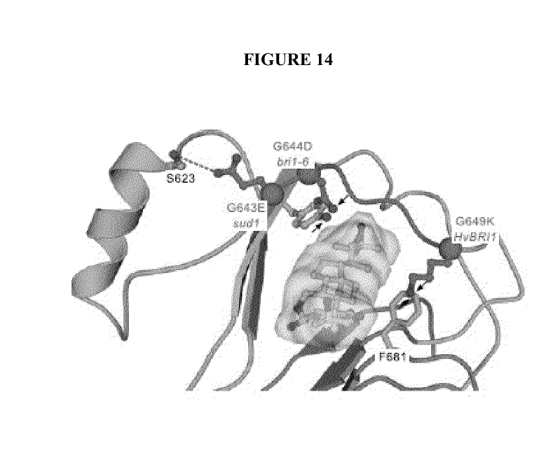

Four known BRI1 missense alleles map to the inner surface of last five LRRs (FIG. 13a). This surface is not masked by carbohydrate and contains both the hormone-binding site and the island domain (FIG. 9a,d and FIG. 12a,c). Three mutations cluster in a loop connecting the island domain with LRR 22 (FIG. 13a). This loop is partially disordered in the unliganded structure but is well-defined in the brassinolide complex (FIG. 10). The results indicate that this loop, when ordered, is engaged in protein-protein interactions that are critical for receptor activation, and that three missense mutations in BRI1 can modulate these interactions. The gain-of-function allele sud1 (Gly643-Glu) likely contacts with Ser623 in the island domain, and leads to an ordered loop even in the absence of steroid ligand (FIG. 14). Mutation of the neighboring Gly644 to Asp causes the loss-of-function phenotype 6 (see above, FIG. 9d, and FIG. 13a), and mutation of conserved Thr649 to Lys inactivates barley BRI1. These mutations, when modeled, induce steric clashes with residues in the island domain and in the underlying LRR domain (FIG. 14), and thus would distort the position of the loop. Interestingly, bri1-102, a strong loss-of-function mutation (Thr750-Ile) (Friedrichsen, Plant Physiol. 123, 1247-1256 (2000)) that does not affect steroid binding, maps to a distinct surface area in LRR 25 (FIG. 13a). The protein-protein interactions involved receptor activation thus likely include residues from the LRR core.

### G. Example 6

### Receptor Activation

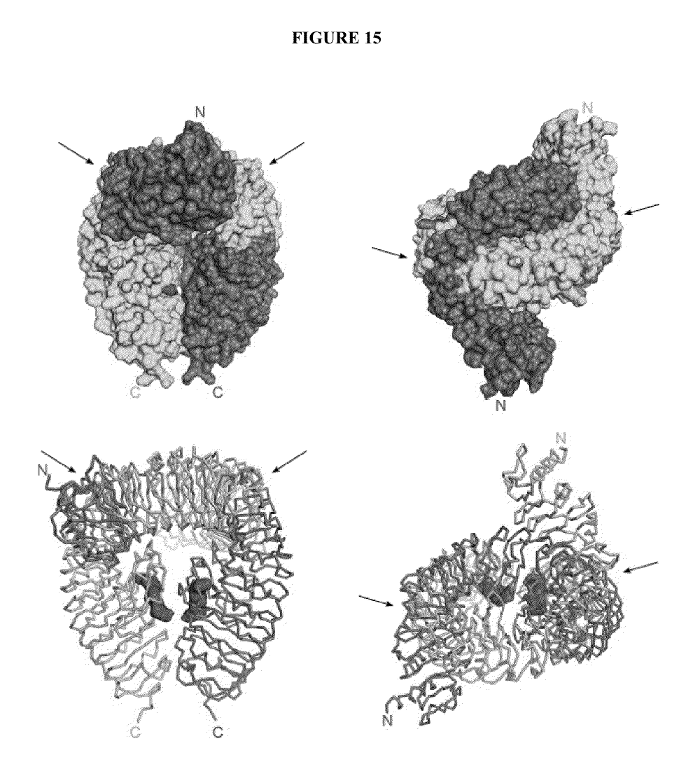

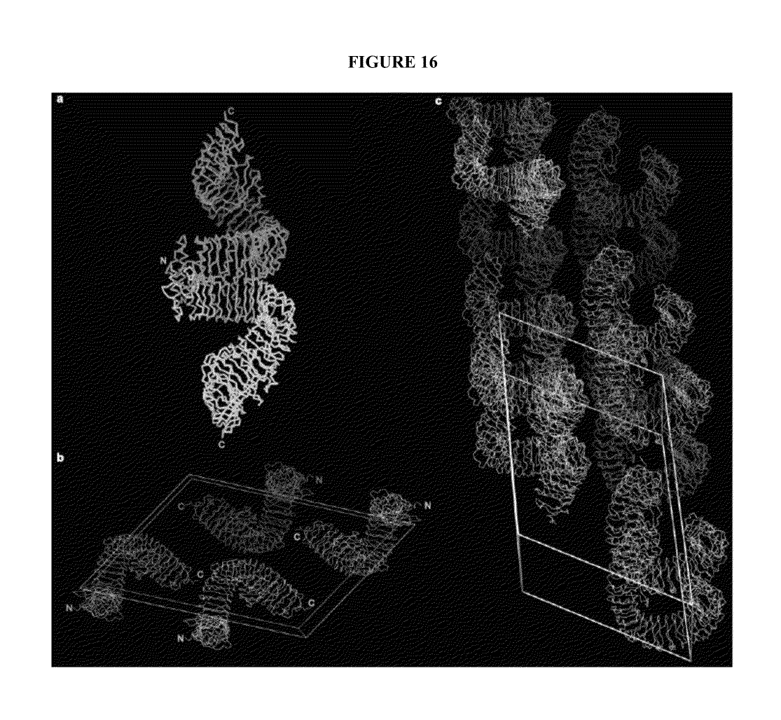

BRI1 has been reported to form homooligomers in plants (Wang et al., Dev. Cell 8, 855-865 (2005); Hink, Biophys. J. 94, 1052-1062 (2008); Russinova et al., Plant Cell 16, 3216-3229 (2004)). The steroid binding to the island domain and the concomitant rearrangements of the island domain loop could induce a conformational change in a preformed BRI1 homodimer, or allow for ligand-dependent dimerization of the BRI1 ectodomain. However, models of BRI1 dimers that bring the C-termini of their ectodomains into close proximity encounter steric clashes with the N-terminal LRRs (FIG. 15). Furthermore, in contrast to TLR ectodomain crystals, which tend to form homodimers even in the absence of ligand, dimers are not observed in BRI1 crystals grown the same acidic pH conditions associated with the plant cell wall. The largest interface area between two neighboring BRI1 molecules amounts to only ˜1.5% of the total accessible surface area, consistent with the high solvent content of the crystals. The main crystal contact involves a head-to-head arrangement of two BRI1 monomers, a configuration that places the cytoplasmic kinase domains far apart (FIG. 16a). No other crystal contacts between neighboring molecules involve either the hormone binding sites or the island domains (FIG. 16a-c). The recombinant BRI1 ectodomain elutes as a monomer in the absence of steroid ligand, and shows no tendency to dimer- or oligomerize in the presence of a ˜4× molar excess of brassinolide in size-exclusion chromatography experiments (FIG. 13b).

The present analyses show that the superhelical BRI1 LRR domain alone has no tendency to oligomerize, indicating that BRI1 receptor activation is not be mediated by ligand-induced homodimerization of the ectodomain (as described for TLRs) or by conformational changes in preformed homodimers. The present structures indicate that homooligomerization of BRI1 is constitutive on some level, and independent of ligand stimulus. The presence of an interaction platform that undergoes conformational changes upon steroid binding, and that harbors several loss- and gain-of-function alleles, indicates that another factor controls activation of BRI1.

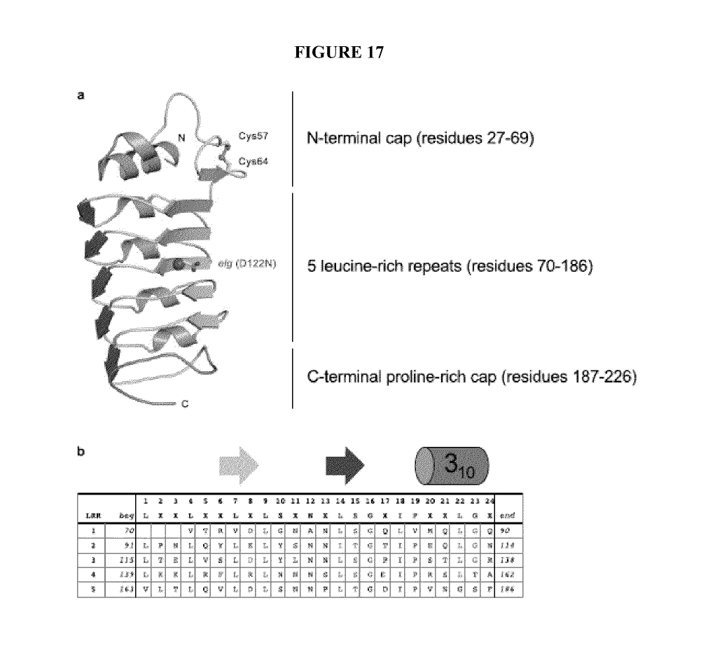

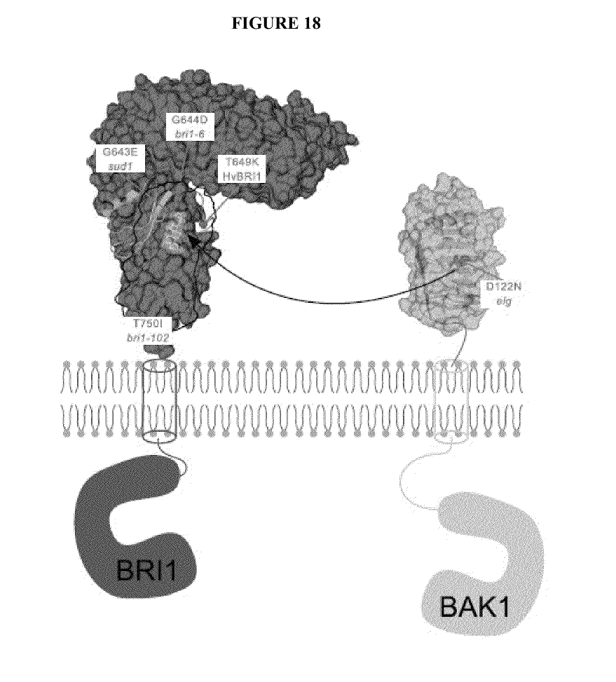

The present results indicate that the superhelical shape of the BRI1 ectodomain is incompatible with homodimerization, and that the isolated ectodomain behaves as a monomer even in the presence of steroid. This finding indicates that another protein factor binds to the interaction platform in BRI1, e.g., encompassing the steroid ligand, LRRs 21-25, and parts of the island domain (FIG. 13a). Genetic and biochemical screens have been carried out for BRI1, but have not uncovered a new protein. The present results indicate that the small receptor kinase BAK1 is likely to act as a direct brassinosteroid co-receptor. BAK1 is a genetic component of the brassinosteroid pathway, BRI1 and BAK1 interact in a steroid-dependent manner, and both receptors trans-phosphorylate each other upon ligand stimulus. Notably, a homology model of the small BAK1 ectodomain (FIG. 17) is compatible in size and shape with the interaction platform in BRI1, and the BAK1 elg allele (Halliday et al., Plant J. 9, 305-312 (1996)), which maps to the inner surface of the BAK1 ectodomain (FIG. 18), renders plants hypersensitive to brassinosteroid treatment. These results indicate that the sud1, bri1-6, bri1-102 and elg mutations modulate the interaction between the BRI1 and BAK1 ectodomains in a brassinosteroid-dependent manner (FIG. 18).

At least two BAK1-like proteins interact with BRI1 in vivo (He et al., Curr. Biol. 17, 1109-1115 (2007); Karlova et al., Plant Cell 18, 626-638 (2006)). The BRI1 inhibitor protein BKI1 blocks the interaction between the BAK1 and BRI1 kinase domains (Jaillais et al., Genes Dev. 25, 232-237 (2011)). In addition, transgenic lines that constitutively deliver BKI1 to the site of BRI1 signaling resemble strong BRI1 loss-of-function mutants. The results support the role of BAK1 in co-activating BRI1.

### Informal Sequence Listing

### BRI1 (Arabidopsis)

(SEQ ID NO: 1)
MKTFSSFFLSVTTLFFFSFFSLSFQASPSQSLYREIHQLISFKDVLPDKNLLPDWSSNKNPCTFDGVTCRDDKVTSI
DLSSKPLNVGFSAVSSSLLSLTGLESLFLSNSHINGSVSGFKCSASLTSLDLSRNSLSGPVTTLTSLGSCSGLKFLN
VSSNTLDFPGKVSGGLKLNSLEVLDLSANSISGANVVGWVLSDGCGELKHLAISGNKISGDVDVSRCVNLEFLDVSS
NNFSTGIPFLGDCSALQHLDISGNKLSGDFSRAISTCTELKLLNISSNQFVGPIPPLPLKSLQYLSLAENKFTGEIP
DFLSGACDTLTGLDLSGNHFYGAVPPFFGSCSLLESLALSSNNFSGELPMDTLLKMRGLKVLDLSFNEFSGELPESL
TNLSASLLTLDLSSNNFSGPILPNLCQNPKNTLQELYLQNNGFTGKIPPTLSNCSELVSLHLSFNYLSGTIPSSLGS
LSKLRDLKLWLNMLEGEIPQELMYVKTLETLILDFNDLTGEIPSGLSNCTNLNWISLSNNRLTGEIPKWIGRLENLA
ILKLSNNSFSGNIPAELGDCRSLIWLDLNTNLFNGTIPAAMFKQSGKIAANFIAGKRYVYIKNDGMKKECHGAGNLL
EFQGIRSEQLNRLSTRNPCNITSRVYGGHTSPTFDNNGSMMFLDMSYNMLSGYIPKEIGSMPYLFILNLGHNDISGS
IPDEVGDLRGLNILDLSSNKLDGRIPQAMSALTMLTEIDLSNNNLSGPIPEMGQFETFPPAKFLNNPGLCGYPLPRC
DPSNADGYAHHQRSHGRRPASLAGSVAMGLLFSFVCIFGLILVGREMRKRRRKKEAELEMYAEGHGNSGDRTANNTN
WKLTGVKEALSINLAAFEKPLRKLTFADLLQATNGFHNDSLIGSGGFGDVYKAILKDGSAVAIKKLIHVSGQGDREF
MAEMETIGKIKHRNLVPLLGYCKVGDERLLVYEFMKYGSLEDVLHDPKKAGVKLNWSTRRKIAIGSARGLAFLHHNC
SPHIIHRDMKSSNVLLDENLEARVSDFGMARLMSAMDTHLSVSTLAGTPGYVPPEYYQSFRCSTKGDVYSYGVVLLE
LLTGKRPTDSPDFGDNNLVGWVKQHAKLRISDVFDPELMKEDPALEIELLQHLKVAVACLDDRAWRRPTMVQVMAMF
KEIQAGSGIDSQSTIRSIEDGGFSTIEMVDMSIKEVPEGKL

1. An isolated protein comprising a 3-dimensional crystal structure of BRassinosteroid Insensitive 1 (BRI1) ectodomain being structurally defined by the atomic coordinate data shown in Tables 1 and 2.

2. An isolated protein comprising a 3-dimensional structure of the BRassinosteroid Insensitive 1 (BRI1) ectodomain, with a space group C2 and unit cell dimensions a=175.09±0.1 angstrom, b=67.25±0.1 angstrom, c=119.05±0.1, with beta=121.55±0.1.

3. An isolated protein comprising a 3-dimensional structure of the BRassinosteroid Insensitive 1 (BRI1) ectodomain being structurally defined by the diagrams shown in FIG. 1 and FIG. 9.

4. The protein of claim 1, wherein the protein comprises a sequence having at least 90% identity to residues 29-788 of SEQ ID NO:1.

5. The protein of claim 1, wherein the protein binds to brassinolide.

6. A method of identifying a candidate modulator of BRassinosteroid Insensitive 1 (BRI1), comprising
(a) comparing the structure of a test compound with the structure of BRI1, said BRI1 comprising a 3 dimensional structure selected from the group consisting of:
(i) an ectodomain structurally defined by the atomic coordinate data shown in Tables 1 and 2;
(ii) a space group C2 and unit cell dimensions a=175.09±0.1 angstrom, b=67.25±0.1 angstrom, c=119.05±0.1, with beta=121.55±0.1; and
(iii) an ectodomain structurally defined by the diagrams shown in FIG. 1 and FIG. 9;
(b) determining whether the test compound is likely to interact with BRI1; and
(c) identifying a candidate BRI1 modulator when the test compound in step (b) is determined to be likely to interact with BRI1.

7. The method of claim 6, further comprising validating the candidate BRI1 modulator by contacting the candidate BRI1 modulator with BRI1 and detecting interaction of the candidate BRI1 modulator with BRI1.

8. The method of claim 6, further comprising detecting an effect of the candidate BRI1 modulator when contacted with a BRI1 expressing plant, wherein the effect is selected from the group consisting of:
increasing plant biomass,
reducing plant biomass,
increasing the size of vegetative structures, and
reducing the size of vegetative structures,
as compared to a standard control.

9. The method of claim 6, wherein the candidate BRI1 modulator interacts with the ligand-binding region of BRI1.

10. The method of claim 6, wherein the candidate BRI1 modulator interacts with the co-receptor interaction region of BRI1.

11. A method of identifying a candidate modulator of BRassinosteroid Insensitive 1 (BRI1), comprising
(a) contacting a test compound with BRI1, said BRI1 comprising a 3 dimensional structure selected from the group consisting of:
(i) an ectodomain structurally defined by the atomic coordinate data shown in Tables 1 and 2;
(ii) a space group C2 and unit cell dimensions a=175.09±0.1 angstrom, b=67.25±0.1 angstrom, c=119.05±0.1, with beta=121.55±0.1; and
(iii) an ectodomain structurally defined by the diagrams shown in FIG. 1 and FIG. 9; and
(b) detecting interaction of the test compound with BRI1, thereby identifying a candidate modulator of BRI1.

12. The method of claim 11, further comprising detecting an effect of the candidate BRI1 modulator when contacted with a BRI1 expressing plant, wherein the effect is selected from the group consisting of:
increasing plant biomass,
reducing plant biomass,
increasing the size of vegetative structures, and
reducing the size of vegetative structures,
as compared to a standard control.

13. The method of claim 11, wherein the candidate BRI1 modulator interacts with the ligand binding region of BRI1.

14. The method of claim 11, wherein the candidate BRI1 modulator interacts with the co-receptor interaction region of BRI1.

15. The protein of claim 2, wherein the protein comprises a sequence having at least 90% identity to residues 29-788 of SEQ ID NO:1.

16. The protein of claim 2, wherein the protein binds to brassinolide.

17. The protein of claim 3, wherein the protein comprises a sequence having at least 90% identity to residues 29-788 of SEQ ID NO:1.

18. The protein of claim 3, wherein the protein binds to brassinolide.


SEQUENCE LISTING (56 sequences)

SEQ ID NO: 1 - protein, 1196 aa, Arabidopsis thaliana
MKTFSSFFLSVTTLFFFSFFSLSFQASPSQSLYREIHQLISFKDVLPDKNLLPDWSSNKNPCTFDGVTCRDDKVTSIDLSSKPLNVGFSAVSSSLLSLTGLESLFLSNSHINGSVSGFKCSASLTSLDLSRNSLSGPVTTLTSLGSCSGLKFLNVSSNTLDFPGKVSGGLKLNSLEVLDLSANSISGANVVGWVLSDGCGELKHLAISGNKISGDVDVSRCVNLEFLDVSSNNFSTGIPFLGDCSALQHLDISGNKLSGDFSRAISTCTELKLLNISSNQFVGPIPPLPLKSLQYLSLAENKFTGEIPDFLSGACDTLTGLDLSGNHFYGAVPPFFGSCSLLESLALSSNNFSGELPMDTLLKMRGLKVLDLSFNEFSGELPESLTNLSASLLTLDLSSNNFSGPILPNLCQNPKNTLQELYLQNNGFTGKIPPTLSNCSELVSLHLSFNYLSGTIPSSLGSLSKLRDLKLWLNMLEGEIPQELMYVKTLETLILDFNDLTGEIPSGLSNCTNLNWISLSNNRLTGEIPKWIGRLENLAILKLSNNSFSGNIPAELGDCRSLIWLDLNTNLFNGTIPAAMFKQSGKIAANFIAGKRYVYIKNDGMKKECHGAGNLLEFQGIRSEQLNRLSTRNPCNITSRVYGGHTSPTFDNNGSMMFLDMSYNMLSGYIPKEIGSMPYLFILNLGHNDISGSIPDEVGDLRGLNILDLSSNKLDGRIPQAMSALTMLTEIDLSNNNLSGPIPEMGQFETFPPAKFLNNPGLCGYPLPRCDPSNADGYAHHQRSHGRRPASLAGSVAMGLLFSFVCIFGLILVGREMRKRRRKKEAELEMYAEGHGNSGDRTANNTNWKLTGVKEALSINLAAFEKPLRKLTFADLLQATNGFHNDSLIGSGGFGDVYKAILKDGSAVAIKKLIHVSGQGDREFMAEMETIGKIKHRNLVPLLGYCKVGDERLLVYEFMKYGSLEDVLHDPKKAGVKLNWSTRRKIAIGSARGLAFLHHNCSPHIIHRDMKSSNVLLDENLEARVSDFGMARLMSAMDTHLSVSTLAGTPGYVPPEYYQSFRCSTKGDVYSYGVVLLELLTGKRPTDSPDFGDNNLVGWVKQHAKLRISDVFDPELMKEDPALEIELLQHLKVAVACLDDRAWRRPTMVQVMAMFKEIQAGSGIDSQSTIRSIEDGGFSTIEMVDMSIKEVPEGKL

SEQ ID NO: 2 - protein, 24 aa, Artificial Sequence
  Synthetic polypeptide
LXXLXXLXLXXNXLSGXIPXXLGX

SEQ ID NO: 3 - protein, 24 aa, Arabidopsis thaliana
CSELVSLHLSFNYLSGTIPSSLGS

SEQ ID NO: 4 - protein, 24 aa, Arabidopsis thaliana
MPYLFILNLGHNDISGSIPDEVGD

SEQ ID NO: 5 - protein, 24 aa, Arabidopsis thaliana
LTELVSLDLYLNNLSGPIPSTLGR

SEQ ID NO: 6 - protein, 24 aa, Arabidopsis thaliana
LVSLKSLDLSINQLTGEIPQSFIN

SEQ ID NO: 7 - protein, 24 aa, Arabidopsis thaliana
CSSLVQLELYDNQLTGKIPAELGN

SEQ ID NO: 8 - protein, 24 aa, Arabidopsis thaliana
LLNLQVVDLYSNAISGEIPSYFGN

SEQ ID NO: 9 - protein, 24 aa, Arabidopsis thaliana
CGSLIKIDLSRNRVTGPIPESINR

SEQ ID NO: 10 - protein, 24 aa, Phaseolus vulgaris
LTQLHYLYITHTNVSGAIPDFLSQ

SEQ ID NO: 11 - protein, 744 aa, Solarium lycopersicum
VNGLYKDSQQLLSFKAALPPTPTLLQNWLSSTGPCSFTGVSCKNSRVSSIDLSNTFLSVDFSLVTSYLLPLSNLESLVLKNANLSGSLTSAAKSQCGVTLDSIDLAENTISGPISDISSFGVCSNLKSLNLSKNFLDPPGKEMLKAATFSLQVLDLSYNNISGFNLFPWVSSMGFVELEFFSLKGNKLAGSIPELDFKNLSYLDLSANNFSTVFPSFKDCSNLQHLDLSSNKFYGDIGSSLSSCGKLSFLNLTNNQFVGLVPKLPSESLQYLYLRGNDFQGVYPNQLADLCKTVVELDLSYNNFSGMVPESLGECSSLELVDISYNNFSGKLPVDTLSKLSNIKTMVLSFNKFVGGLPDSFSNLLKLETLDMSSNNLTGVIPSGICKDPMNNLKVLYLQNNLFKGPIPDSLSNCSQLVSLDLSFNYLTGSIPSSLGSLSKLKDLILWLNQLSGEIPQELMYLQALENLILDFNDLTGPIPASLSNCTKLNWISLSNNQLSGEIPASLGRLSNLAILKLGNNSISGNIPAELGNCQSLIWLDLNTNFLNGSIPPPLFKQSGNIAVALLTGKRYVYIKNDGSKECHGAGNLLEFGGIRQEQLDRISTRHPCNFTRVYRGITQPTFNHNGSMIFLDLSYNKLEGSIPKELGAMYYLSILNLGHNDLSGMIPQQLGGLKNVAILDLSYNRFNGTIPNSLTSLTLLGEIDLSNNNLSGMIPESAPFDTFPDYRFANNSLCGYPLPIPCS

SEQ ID NO: 12 - protein, 744 aa, Glycine max
FSSSSPVTQQLLSFKNSLPNPSLLPNWLPNQSPCTFSGISCNDTELTSIDLSSVPLSTNLTVIASFLLSLDHLQSLSLKSTNLSGPAAMPPLSHSQCSSSLTSLDLSQNSLSASLNDMSFLASCSNLQSLNLSSNLLQFGPPPHWKLHHLRFADFSYNKISGPGVVSWLLNPVIELLSLKGNKVTGETDFSGSISLQYLDLSSNNFSVTFPTFGECSSLEYLDLSANKYLGDIARTLSPCKSLVYLNVSSNQFSGPVPSLPSGSLQFVYLAANHFHGQIPLSLADLCSTLLQLDLSSNNLTGALPGAFGACTSLQSLDISSNLFAGALPMSVLTQMTSLKELAVAFNGFLGALPESLSKLSALELLDLSSNNFSGSIPASLCGGGDAGINNNLKELYLQNNRFTGFIPPTLSNCSNLVALDLSFNFLTGTIPPSLGSLSNLKDFIIWLNQLHGEIPQELMYLKSLENLILDFNDLTGNIPSGLVNCTKLNWISLSNNRLSGEIPPWIGKLSNLAILKLSNNSFSGRIPPELGDCTSLIWLDLNTNMLTGPIPPELFKQSGKIAVNFISGKTYVYIKNDGSKECHGAGNLLEFAGISQQQLNRISTRNPCNFTRVYGGKLQPTFNHNGSMIFLDISHNMLSGSIPKEIGAMYYLYILNLGHNNVSGSIPQELGKMKNLNILDLSNNRLEGQIPQSLTGLSLLTEIDLSNNLLTGTIPESGQFDTFPAAKFQNNSGLCGVPLGPCG

SEQ ID NO: 13 - protein, 742 aa, Nicotiana tabacum
VNGLFKDSQQLLSFKSSLPNTQTQLQNWLSSTDPCSFTGVSCKNSRVSSIDLTNTFLSVDFTLVSSYLLGLSNLESLVLKNANLSGSLTSAAKSQCGVSLNSIDLAENTISGPVSDISSFGACSNLKSLNLSKNLMDPPSKELKASTFSLQDLDLSFNNISGQNLFPWLSSMRFVELEYFSVKGNKLAGNIPELDFTNLSYLDLSANNFSTGFPSFKDCSNLEHLDLSSNKFYGDIGASLSSCGKLSFLNLTNNQFVGLVPKLPSESLQFLYLRGNDFQGVFPSQLADLCKTLVELDLSFNNFSGLVPENLGACSSLEFLDISNNNFSGKLPVDTLLKLSNLKTMVLSFNNFIGGLPESFSNLLKLETLDVSSNNITGFIPSGICKDPMSSLKVLYLQNNWFTGPIPDSLSNCSQLVSLDLSFNYLTGKIPSSLGSLSKLKDLILWLNQLSGEIPQELMYLKSLENLILDFNDLTGSIPASLSNCTNLNWISMSNNLLSGEIPASLGGLPNLAILKLGNNSISGNIPAELGNCQSLIWLDLNTNFLNGSIPGPLFKQSGNIAVALLTGKRYVYIKNDGSKECHGAGNLLEFGGIRQEQLDRISTRHPCNFTRVYRGITQPTFNHNGSMIFLDLSYNKLEGGIPKELGSMYYLSILNLGHNDFSGVIPQELGGLKNVAILDLSYNRLNGSIPNSLTSLTLLGELDLSNNNLTGPIPESAPFDTFPDYRFANTSLCGYPLQPCG

SEQ ID NO: 14 - protein, 677 aa, Oryza sativa
GAAAADDAQLLEEFRQAVPNQAALKGWSGGDGACRFPGAGCRNGRLTSLSLAGVPLNAEFRAVAATLLQLGSVEVLSLRGANVSGALSAAGGARCGSKLQALDLSGNAALRGSVADVAALASACGGLKTLNLSGDAVGAAKVGGGGGPGFAGLDSLDLSNNKITDDSDLRWMVDAGVGAVRWLDLALNRISGFPEFTNCSGLQYLDLSGNLIVGEVPGGALSDCRGLKVLNLSFNHLAGVFPPDIAGLTSLNALNLSNNNFSGELPGEAFAKLQQLTALSLSFNHFNGSIPDTVASLPELQQLDLSSNTFSGTIPSSLCQDPNSKLHLLYLQNNYLTGGIPDAVSNCTSLVSLDLSLNYINGSIPASLGDLGNLQDLILWQNELEGEIPASLSRIQGLEHLILDYNGLTGSIPPELAKCTKLNWISLASNRLSGPIPSWLGKLSYLAILKLSNNSFSGPIPPELGDCQSLVWLDLNSNQLNGSIPKELAKQSGKMNVGLIVGRPYVYLRNDELSSECRGKGSLLEFTSIRPDDLSRMPSKKLCNFTRMYVGSTEYTFNKNGSMIFLDLSYNQLDSAIPGELGDMFYLMIMNLGHNLLSGTIPSRLAEAKKLAVLDLSYNQLEGPIPNSFSALSLSEINLSNNQLNGTIPELGSLATFPKSQYENNTGLCGFPLPPCD

SEQ ID NO: 15 - protein, 730 aa, Arabidopsis thaliana
LSDDVNDTALLTAFKQTSIKSDPTNFLGNWRYGSGRDPCTWRGVSCSSDGRVIGLDLRNGGLTGTLNLNNLTALSNLRSLYLQGNNFSSGDSSSSSGCSLEVLDLSSNSLTDSSIVDYVFSTCLNLVSVNFSHNKLAGKLKSSPSASNKRITTVDLSNNRFSDEIPETFIADFPNSLKHLDLSGNNVTGDFSRLSFGLCENLTVFSLSQNSISGDRFPVSLSNCKLLETLNLSRNSLIGKIPGDDYWGNFQNLRQLSLAHNLYSGEIPPELSLLCRTLEVLDLSGNSLTGQLPQSFTSCGSLQSLNLGNNKLSGDFLSTVVSKLSRITNLYLPFNNISGSVPISLTNCSNLRVLDLSSNEFTGEVPSGFCSLQSSSVLEKLLIANNYLSGTVPVELGKCKSLKTIDLSFNALTGLIPKEIWTLPKLSDLVMWANNLTGGIPESICVDGGNLETLILNNNLLTGSLPESISKCTNMLWISLSSNLLTGEIPVGIGKLEKLAILQLGNNSLTGNIPSELGNCKNLIWLDLNSNNLTGNLPGELASQAGLVMPGSVSGKQFAFVRNEGGTDCRGAGGLVEFEGIRAERLEHFPMVHSCPKTRIYSGMTMYMFSSNGSMIYLDLSYNAVSGSIPLGYGAMGYLQVLNLGHNLLTGTIPDSFGGLKAIGVLDLSHNDLQGFLPGSLGGLSFLSDLDVSNNNLTGPIPFGGQLTTFPLTRYANNSGLCGVPLPPCS

SEQ ID NO: 16 - protein, 45 aa, Arabidopsis thaliana
SQSLYREIHQLISFKDVLPDKNLLPDWSSNKNPCTFDGVTCRDDK

SEQ ID NO: 17 - protein, 47 aa, Phaseolus vulgaris
ELCNPQDKQALLQIKKDLGNPTTLSSWLPTTDCCNRTWLGVLCDTDT

SEQ ID NO: 18 - protein, 47 aa, Arabidopsis thaliana
RVSGNAEGDALSALKNSLADPNKVLQSWDATLVTPCTWFHVTCNSDN

SEQ ID NO: 19 - protein, 48 aa, Arabidopsis thaliana
CFAYTDMEVLLNLKSSMIGPKGHGLHDWIHSSSPDAHCSFSGVSCDDD

SEQ ID NO: 20 - protein, 48 aa, Arabidopsis thaliana
RFSNETDMQALLEFKSQVSENNKREVLASWNHSSPFCNWIGVTCGRRR

SEQ ID NO: 21 - protein, 49 aa, Arabidopsis thaliana
KQSFEPEIEALKSFKNGISNDPLGVLSDWTIIGSLRHCNWTGITCDSTG

SEQ ID NO: 22 - protein, 42 aa, Arabidopsis thaliana
RTEPDEQDAVYDIMRATGNDWAAAIPDVCRGRWHGIECMPDG

SEQ ID NO: 23 - protein, 41 aa, Arabidopsis thaliana
FETFPPAKFLNNPGLCGYPLPRCDPSNADGYAHHQRSHGRR

SEQ ID NO: 24 - protein, 24 aa, Phaseolus vulgaris
LQRFDVSAYANNKCLCGSPLPACT

SEQ ID NO: 25 - protein, 34 aa, Arabidopsis thaliana
FLVFNETSFAGNTYLCLPHRVSCPTRPGQTSDHN

SEQ ID NO: 26 - protein, 39 aa, Arabidopsis thaliana
FKNINASDLMGNTDLCGSKKPLKPCTIKQKSSHFSKRTR

SEQ ID NO: 27 - protein, 19 aa, Arabidopsis thaliana
HCHDPVAHCDIKPSNILLD

SEQ ID NO: 28 - protein, 21 aa, Arabidopsis thaliana
VTRVDLGNANLSGQLVMQLGQ

SEQ ID NO: 29 - protein, 24 aa, Arabidopsis thaliana
LPNLQYLELYSNNITGTIPEQLGN

SEQ ID NO: 30 - protein, 24 aa, Arabidopsis thaliana
LKKLRFLRLNNNSLSGEIPRSLTA

SEQ ID NO: 31 - protein, 24 aa, Arabidopsis thaliana
VLTLQVLDLSNNPLTGDIPVNGSF

SEQ ID NO: 32 - protein, 23 aa, Artificial Sequence
  Synthetic polypeptide
VTSIDLSSKPLNVGFSAVSSLLS

SEQ ID NO: 33 - protein, 23 aa, Arabidopsis thaliana
LTGLESLFLSNSHINGSVSGFKC

SEQ ID NO: 34 - protein, 26 aa, Artificial Sequence
  Synthetic polypeptide
SASLTSLDLCRNSLSGPVTTLTSLGS

SEQ ID NO: 35 - protein, 25 aa, Arabidopsis thaliana
CSGLKFLNVSSNTLDFPGKVSGGLK

SEQ ID NO: 36 - protein, 27 aa, Arabidopsis thaliana
LNSLEVLDLSANSISGANVVGWVLSDG

SEQ ID NO: 37 - protein, 20 aa, Artificial Sequence
  Synthetic polypeptide
CGELKHLAISGNKISGDVSR

SEQ ID NO: 38 - protein, 23 aa, Artificial Sequence
  Synthetic polypeptide
CVNLEFLEVSSNNFSTGIPFLGD

SEQ ID NO: 39 - protein, 24 aa, Arabidopsis thaliana
CSALQHLDISGNKLSGDFSRAIST

SEQ ID NO: 40 - protein, 22 aa, Arabidopsis thaliana
CTELKLLNISSNQFVGPIPPLP

SEQ ID NO: 41 - protein, 25 aa, Arabidopsis thaliana
LKSLQYLSLAENKFTGEIPDFLSGA

SEQ ID NO: 42 - protein, 24 aa, Arabidopsis thaliana
CDTLTGLDLSGNHFYGAVPPFFGS

SEQ ID NO: 43 - protein, 25 aa, Arabidopsis thaliana
CSLLESLALSSNNFSGELPMDTLLK

SEQ ID NO: 44 - protein, 24 aa, Arabidopsis thaliana
MRGLKVLDLSFNEFSGELPESLTN

SEQ ID NO: 45 - protein, 24 aa, Arabidopsis thaliana
LSASLLTLDLSSNNFSGPILPNLC

SEQ ID NO: 46 - protein, 23 aa, Arabidopsis thaliana
NTLQELYLQNNGFTGKIPPTLSN

SEQ ID NO: 47 - protein, 25 aa, Artificial Sequence
  Synthetic polypeptide
CSELVSLHLSFNYLSGTIPSSSLGS

SEQ ID NO: 48 - protein, 24 aa, Arabidopsis thaliana
LSKLRDLKLWLNMLEGEIPQELMY

SEQ ID NO: 49 - protein, 24 aa, Artificial Sequence
  Synthetic polypeptide
VKTLETLILDFNELTGEIPSGLSN

SEQ ID NO: 50 - protein, 24 aa, Arabidopsis thaliana
CTNLNWISLSNNRLTGEIPKWIGR

SEQ ID NO: 51 - protein, 24 aa, Arabidopsis thaliana
LENLAILKLSNNSFSGNIPAELGD

SEQ ID NO: 52 - protein, 25 aa, Arabidopsis thaliana
CRSLIWLDLNTNLFNGTIPAAMFKQ

SEQ ID NO: 53 - protein, 22 aa, Arabidopsis thaliana
SMMFLDMSYNMLSGYIPKEIGS

SEQ ID NO: 54 - protein, 24 aa, Arabidopsis thaliana
MPYLFILNLGHNDISGSIPDEVGD

SEQ ID NO: 55 - protein, 24 aa, Arabidopsis thaliana
LRGLNILDLSSNKLDGRIPQAMSA

SEQ ID NO: 56 - protein, 24 aa, Artificial Sequence
  Synthetic polypeptide
LTMLTEIDLSNNNLSGRIPQAMGQ